(19) United States
(12) Patent Application Publication
(10) Pub. No.: US 2026/0219156 A1
(43) Pub. Date: Jul. 30, 2026

(54) CHARACTERIZING AEROSOL PARTICLES USING PNEUMATIC-NEBULIZER-INDUCED DISINTEGRATION

(71) Applicant: LIGHT MATTER INTERACTION INC., ETOBICOKE (CA)

(72) Inventor: KRESIMIR FRANJIC, TORONTO (CA)

(21) Appl. No.: 19/148,802

(22) PCT Filed: Jan. 18, 2024
(86) PCT No.: PCT/CA2024/050054

§ 371 Date: Jul. 17, 2025

(30) Foreign Application Priority Data
Jan. 18, 2023 (CA) 3186817

(51) Int. Cl. G01N 15/14; A61B 18/00; A61B 18/14; A61B 18/20; B23K 26/36; G01N 1/28; G01N 1/40; G01N 29/02; G01N 29/24; H01J 49/04

(52) U.S. Cl. CPC G01N 15/14; A61B 18/14; A61B 18/20; B23K 26/36; G01N 1/286; G01N 1/4077; G01N 29/02; G01N 29/2418; H01J 49/0422; A61B 2018/00577; G01N 2001/2886; G01N 2001/4038; G01N 2001/4088

(57) ABSTRACT

Systems and methods are provided for disintegration and analysis of analyte aerosol particles. In various embodiments, these systems collect aerosol particles and introduce them into a high-pressure gas stream. This stream, upon ejection near the exit of a liquid conduit, nebulizes the exiting liquid, facilitating high-impact collisions between the aerosol particles and the liquid. This interaction leads to the disintegration, complete or partial, of the aerosol particles, with the potential for their dissolution in the resulting liquid droplets. In certain embodiments, at least some of the resulting analyte species are ions that can be analyzed with a mass spectrometer. These systems and methods are adaptable for analyzing a broad range of aerosols and especially hold promise for examining plumes generated through laser ablation of tissues.

### RELATED APPLICATION

This application claims priority to Canadian Patent Application No. 3, 186,817, titled “MOLECULAR CHARACTERIZATION OF AEROSOL PARTICLES THROUGH PNEUMATIC NEBULIZER ASSISTED FRAGMENTATION” and filed on Jan. 18, 2023, which is incorporated herein by reference in its entirety.

### BACKGROUND

Analyzing the chemical composition of aerosols is vital across various scientific fields, environmental monitoring, medicine, and industry. Aerosol analysis is crucial not only for its intrinsic value but also for understanding the sources of aerosols and their interactions with different systems. For instance, the characterization of bioaerosols (like fungi, pollen, spores, viruses, bacteria) in air and human environments is critically important. Another key area is the analysis of laser plumes generated by the ablation of biological tissues. Recent advances have shown that infrared laser pulses with short durations (around 0.1-5 ns) and wavelengths near 2.94 μm, resonating with O—H stretch vibrations of water, can cut biological tissues with minimal collateral damage [1]. Such laser ablation acts by creating ultrafast acoustic transients in tissues through strongly localized optical absorption centers, micro-cavitations, and acoustic impedance mismatches [2]. As a result, the ejected plume predominantly contains tissue fragments with their molecular composition largely preserved, offering an excellent opportunity for rapid chemical characterization. This method, for example, has potential applications in intraoperative molecular biopsies and tissue imaging [3]. However, a challenge arises as only a fraction of the laser plume is composed of individual molecules or molecular clusters, with the majority being aerosol particles of various sizes [4]. Although some molecular information can be gathered from analysis of the small amount of individual molecular species that get ejected directly from tissues [5], enhancing the extraction of molecules from these aerosol particles for analysis could significantly improve the signal-to-noise ratio for molecular characterization.

Aerosol particles can be analyzed either off-line or in real-time. Off-line techniques typically involve trapping aerosol particles in a liquid or solid matrix for subsequent analysis [6], [7]. While these methods accomplish chemical characterization, they are labor-intensive and time-consuming. Applications like environmental monitoring, intraoperative surgical tissue biopsies, or tissue imaging necessitate real-time analysis, particularly challenging for fragile constituents such as biomolecules [8], [9].

To address this need, several approaches for rapid molecular extraction from aerosol particles have been explored.

Thermal approaches involve heating aerosol particles to desorb molecular constituents through evaporation or thermal disintegration [10], [11]. However, this method may not be suitable for thermally sensitive molecules or those strongly bonded to aerosol particles.

In contrast, dissolution approaches entail blending analyte aerosol particles with a liquid solvent aerosol. This combination results in the merging and partial dissolution of aerosol molecules within the solvent. [12], [13], [14]. As the solvent droplets subsequently evaporate, the dissolved molecules are liberated. However, given that both the original and solvent aerosols are dispersed in a gas, the frequency of aerosol merging is relatively low. Additionally, the interaction time between aerosols is often too brief to allow for complete dissolution of the particles.

Another method is collision approaches, where aerosol particles are mechanically impacted against a hard surface, causing some molecules to eject into the surroundings.

While combining this with dissolution enhances molecular extraction, [15], [16], [17], it necessitates complex modifications to the mass spectrometer interface and does not allow for easy additional ionization. Moreover, frequent impacts of wet aerosols can leave residues, leading to cross-contamination and maintenance challenges.

Given these limitations, there is a clear need for innovative systems and methods capable of extracting molecular species from aerosol particles quickly and gently.

Once separated from the aerosol particles, individual analyte molecular species can be characterized using various analytical tools. Mass spectrometry is particularly effective for this purpose, as it rapidly provides detailed molecular information about complex samples. An additional ionization step of the desorbed aerosol molecules is required for mass spectrometry analysis. Over the past thirty years, significant advancements have been made in developing soft atmospheric pressure ionization methods suitable for real-time analysis [18].

In some scenarios, optical analysis of the desorbed aerosol molecules may also be beneficial.

The method of aerosol particle analysis involves deciding where to sample, modify, and analyze these particles. For mass spectrometry, one option involves transporting aerosol particles through a gas transport line near the mass spectrometer for ionization [19]. Alternatively, ions can be generated away from the mass spectrometer and transported via an ion transport line [20].

While both methods are effective, practical considerations sometimes favor keeping ion production separate from the mass spectrometer to reduce the need for frequent cleaning and maintenance of its interfaces. However, in cases like inductively coupled plasma (ICP) mass spectrometry, ion creation near the mass spectrometer is necessary due to the nature of the ionization process. Therefore, having aerosol disintegration devices that offer flexibility in ion production locations and transport arrangements is important, allowing for optimization based on various application requirements.

### SUMMARY

Aerosols consist of fine solid particles or liquid droplets suspended in a gas and can originate from natural or anthropogenic sources. For clarity, the term “aerosol particles” in this disclosure refers to both solid particles and liquid droplets. Typically, these particles range in size from 1 nm to 100 μm and include various forms such as laser plumes, fumes, smokes, bioaerosols, pollution particulates, and other types found in environmental, medical, agricultural, and industrial contexts.

For the purpose of analysis, the term ‘sample aerosol particles’ will be used to describe any aerosol particles ready for capture by an aspirator device. Aerosol particles are subject to various disintegration processes, and we define specific outcomes of these processes for clarity. “Aerosol particle fragments” result from the breaking of aerosol particles into smaller pieces, while “aerosol molecular species” denotes individual molecules or molecular clusters that detach from an aerosol particle or its fragments through collision, desorption, or dissolution. The collective of aerosol particle fragments and aerosol molecular species and intact particles is termed “aerosol material”. Finally, when aerosol molecular species become charged, they are referred to as “aerosol ions”.

This disclosure centers around a method where sample aerosol particles are introduced into a high-pressure gas stream, which is then directed onto a liquid volume. The impact of the gas causes nebulization, breaking the liquid into droplets, either completely or partially. During this nebulization, the aerosol particles undergo strong collisions with the liquid, leading to their partial or complete disintegration. The gas responsible for transporting the aerosol particles and facilitating nebulization is referred to as “the nebulizing gas”.

When the pressure of the nebulizing gas within its conduit is high enough (usually several bars), the mechanical forces generated during nebulization are substantial. In typical nebulizer devices used for liquid chromatography-mass spectrometry (LC-MS), nebulizing gas can reach impact velocities of 100-300 m/s. Notably, the gas decelerates almost completely within just a few millimeters from the gas nozzle exit, primarily due to collisions with the liquid and ensuing turbulence [21]. This rapid deceleration, on the order of 340,000 g, transfers enough momentum from the gas to the liquid to create a fine spray at rates up to approximately 1 mL/min. It's noteworthy that despite these intense collisions, the individual molecules of liquid analytes in LC-MS usually remain intact during nebulization, as molecular fragmentation can result in the loss of original sample information. Nonetheless, controlled fragmentation during these collisions may be advantageous in certain contexts for extracting specific information. Since the degree of molecular fragmentation is linked to the kinetic energy of impact, adjusting the nebulizing gas's kinetic energy allows us to modulate the extent of molecular fragmentation to suit specific analytical needs.

Nebulization offers the added advantage of stable performance across a wide range of parameters, a valuable trait for analytical tools. Historically, nebulizing gases in analytical chemistry have been employed solely for nebulizing liquid-form analytes, rather than for transporting the analytes themselves. This technique is exemplified in devices like mass spectrometers designed for pneumatically assisted electrospray ionization, as well as those based on inductively coupled plasma (ICP) ionization.

When the target liquid is a solvent, the nebulization process not only causes aerosol disintegration through collisions but also facilitates partial or complete dissolution of aerosol particles and fragments upon contact with the liquid. This interaction leads to the detachment of individual molecules or molecular complexes from the aerosol particles and fragments due to solvation, thereby enhancing the disintegration of aerosol particles. While using liquid aerosols for dissolving sample aerosols has been previously reported [14], [15], the present disclosure offers the advantage of mixing analyte aerosol material with a substantially larger quantity of solvent in a more concentrated space. This results in intense mixing, akin to the disintegration observed in ultrasonic baths, thanks to the kinetic energy of nebulization. For instance, in laser ablation electrospray ionization (LAESI) [14], the mixing of the laser plume aerosol with the electrospray occurs within about 1 cm³ of gas volume, requiring the laser ablation spot to be positioned at a distance for gentle mixing. Additionally, small liquid flow rates and precise geometry adjustments are necessary to maintain the electrospray stability. By contrast, according to the systems and methods of the present disclosure, a similar quantity of laser plume can potentially be mixed with 10 to 1000 times more liquid in a 10 to 100 times smaller volume, achieving more thorough mixing due to the nebulization process. This approach allows for greater efficiency and effectiveness in the mixing and analysis process.

In addition to solvation, leveraging other properties of the liquid can further enhance aerosol disintegration or the molecular characterization of aerosols. For instance, heating the liquid can improve solvation rates. A warmer liquid also evaporates more quickly post-spray, speeding up the analyte desorption process. However, it can be beneficial to maintain a temperature that is low enough to prevent unwanted thermal alterations to the aerosol material.

Another potential advantage involves incorporating molecular labels into the liquid. These labels can specifically target aerosol molecular species during and after nebulization, facilitating more effective molecular detection and quantification.

Furthermore, the liquid can include components that facilitate the ionization of aerosol molecular species for mass spectrometry analysis. For instance, the liquid might contain easily ionizable substances like toluene, which can serve as donor molecules in atmospheric pressure photoionization (APPI) mass spectrometry. This approach offers a dual advantage: firstly, the donor substances get thoroughly mixed with the analyte aerosol material during nebulization, and secondly, the combination of analyte material with the donor gas occurs in a confined space. This compact mixing can enhance ionization efficiency by allowing for application of more focused UV light.

Various nebulizer designs, as known in the field and some of them listed in [22], are compatible with the disclosed embodiments, though specific designs may offer distinct advantages depending on the use case. For instance, introducing nebulizing gas parallel to the liquid conduit outlet can draw the liquid into the gas stream due to Bernoulli's effect, potentially enhancing the interaction between aerosol material and the liquid. Contemporary electrospray ionization (ESI) nebulizers typically feature a liquid capillary concentrically placed within a gas nebulizer capillary. An alternative configuration, where a nebulizing gas capillary is positioned inside a liquid capillary is also possible [22], [23]. This inverted design could be particularly beneficial for our purposes if aerosol particles are introduced through the central nebulizing gas capillary. In such a setup, most collisions between the aerosol particles and the liquid occur within a region engulfed by the liquid drawn into the gas stream. This could lead to more intense collisions and better confinement of the resultant aerosol particle fragments and aerosol molecular species.

In preferred, though not exclusive, embodiments, the nebulization of the liquid takes place within an ambient gas environment at or near standard atmospheric pressure.

For further aerosol disintegration, multiple embodiments of the present disclosure can be configured in a cascading sequence. In this arrangement, the aerosol particle fragments and aerosol molecular species output from one device become the input for the subsequent nebulizer for additional disintegration. This cascading design is also beneficial for multiplexing chemical effects when different solvents are employed in each nebulizer.

After being fragmented into smaller bits, such as particle fragments or molecular species, these aerosol particle fragments and aerosol molecular species can be analyzed using a range of analytical chemistry tools, including optical methods and mass spectrometry. For mass spectrometry analysis, the individual molecular species derived from aerosol particles require ionization. This can be achieved through various methods known in the field. The simplest method mirrors that used in conventional pneumatically assisted electrospray ionization (ESI) devices, where a charged liquid is nebulized. Here, the liquid is charged prior to nebulization, allowing aerosol molecular species to acquire charges either directly during nebulization or subsequently when entrapped in charged droplets that eventually disintegrate due to evaporation or Coulomb explosions, as seen in standard ESI processes. This results in the formation of analyte molecular ions. Other potential ionization methods include atmospheric pressure chemical ionization (APCI) [21], atmospheric pressure photoionization (APPI) [18], [21], [24], and direct photo-ionization [25].

Once ions are generated, they can be gathered and channeled into a mass spectrometer for detailed analysis. However, collecting ions in atmospheric conditions is challenging due to the significant influence of gas streams and electric fields on ion trajectories. Consequently, in conventional ESI devices, ion collection efficiency often diminishes as the pressure of the nebulizing gas increases, complicating the ion corralling process [21]. Any ion collection method familiar to those skilled in the art can be utilized. Ion funnels that operate effectively under atmospheric conditions are particularly advantageous. They can significantly enhance ion sensitivities and detection limits, potentially improving them by 15-100 times [26], [27].

Suitable mass spectrometers for this application encompass any type known to those skilled in the field, with certain types being more apt for specific aerosol analyses. In the case of biological aerosols, mass spectrometers that support soft ionization methods are preferred. These include quadrupole mass spectrometers, time-of-flight (TOF) mass spectrometers, ion trap mass spectrometers, Orbitrap mass spectrometers, triple quadrupole mass spectrometers, Fourier transform ion cyclotron resonance (FT-ICR) mass spectrometers, and various hybrid systems. For investigating sub-molecular compositions of aerosols, techniques like inductively coupled plasma mass spectrometry (ICP-MS) can be particularly useful.

Apart from mass spectrometry, aerosol fragments and molecular species can be examined using any other established analytical method, such as optical spectroscopy analysis, familiar to experts in the field. When dealing with aerosols of complex molecular composition, the multiplexing of diverse complementary analytical methods can be advantageous. This strategy helps reduce data ambiguity, expands observational capabilities, overcomes individual technique limitations, improves quantitative accuracy, speeds up data acquisition and analysis, and supports data cross-validation.

Within the scope of the present disclosure, two exemplary scenarios illustrate the substantial benefits of employing such analytical multiplexing configurations.

Firstly, combining mass spectrometry with vibrational spectroscopy imaging offers a powerful analytical approach if aerosol particles are sampled with a laser. Techniques like spontaneous Raman imaging, stimulated Raman imaging (SRI), or coherent anti-Stokes Raman scattering (CARS) can initially be used to image the sample. These vibrational imaging methods are adept at identifying chemical bond concentrations, characterizing molecular environments, and highlighting inter-molecular interactions. While they may not match the molecule-specific identification capabilities of mass spectrometry, they complement it by providing additional sample attributes. For example, SRI can rapidly distinguish protein-to-lipid ratios, creating color maps similar to histological slides, and thus provide insights into tissue morphology [28]. Optical imaging, with its inherent speed, non-invasiveness, and superior spatial resolution, is ideal for guiding target area selection for mass spectrometry or for aiding the interpretation of mass spectrometry data. On the other hand, mass spectrometry excels in identifying and quantifying specific molecular biomarkers, a feat more challenging for vibrational imaging.

Another promising multiplexing approach is the combination of mass spectrometry with gas chromatography. While mass spectrometry paired with liquid chromatography (LC-MS) is a proven method for analyzing complex samples [29], its relative slowness can be a limitation in applications like tissue imaging, environmental monitoring, or surgical biopsies. A faster alternative is the integration of gas chromatography with mass spectrometry (GC-MS), an approach that has shown potential [30]. In this setup, ions are first collected in an ion trap, then released into a gas-filled drift tube, and finally introduced into a time-of-flight mass spectrometer. The ions are separated based on their drift time through the gas tube and their time of flight, offering a dual-dimension separation. Enhancements like an improved ion gating system could further increase mass-to-charge resolution. This GC-MS technique could be particularly effective for aerosol samples from pulsed laser ablation, as the expansion of the laser plume and collection time (~100 ms) is well-matched with the collection timing of the ion trap, presenting opportunities to enhance signal-to-noise ratios for experts in the field.

The embodiments described in the present disclosure are suitable for analyzing a wide range of aerosols. These include, but not limited to, laser plumes, fumes, smokes, bioaerosols, pollution particulates, natural dust, atmospheric aerosols, as well as pharmaceutical, medical, surgical, agricultural, and industrial aerosols.

Accordingly, in a first aspect, there is provided a system for performing at least partial disintegration of sample aerosol particles, the system comprising:
a liquid source unit comprising a liquid conduit, the liquid conduit comprising a liquid conduit outlet, the liquid source unit being configured to dispense a liquid through the liquid conduit outlet;
a pneumatic nebulizer comprising a high-pressure nebulizing gas mechanism in gas-flow communication with a nebulizing gas conduit, the nebulizing gas conduit comprising a nebulizing gas conduit inlet and a nebulizing gas conduit outlet, wherein the nebulizing gas conduit outlet is positioned adjacent to the liquid conduit outlet to facilitate pneumatic nebulization of the liquid exiting the liquid conduit outlet by contact with a high-pressure nebulizing gas exiting the nebulizing gas conduit;
an aspirator device comprising an aspirator conduit, the aspirator conduit comprising an aspirator conduit inlet and an aspirator conduit outlet, the aspirator device being configured to generate suction suitable for drawing the sample aerosol particles into the aspirator conduit through the aspirator conduit inlet; and
a gas coupler providing gas coupling between the aspirator conduit outlet and the nebulizing gas conduit inlet, thereby facilitating the introduction of the aerosol particles into the high-pressure nebulizing gas stream, such that the nebulization process promotes collisions between the aerosol particles and the liquid, contributing to the disintegration, at least in part, of the aerosol particles.

In some example implementations of the system, the liquid comprises a solvent configured to dissolve a least a portion of the aerosol material either during the nebulization process, within the liquid droplets resulting from the nebulization process, or during both stages.

In some example implementations of the system, the gas coupler includes at least one gas compressor.

In some example implementations of the system, the gas coupler includes at least one Venturi pump.

In some example implementations of the system, the gas coupler includes at least one device selected from the group consisting of: scroll compressor, diaphragm compressor, screw compressor, axial compressor, centrifugal compressor, rotary vane compressor, scroll pump, diaphragm pump, centrifugal pump, and rotary vane pump.

In some example implementations of the system, the system further comprises an additional gas source of an additional gas in fluid communication with the gas coupler, such that the additional gas is introduced into the nebulizing gas conduit and mixed with the aerosol particles.

In some example implementations of the system, the gas coupler comprises one of a gas compressor and a gas pump as a component, and the fluid communication between the additional gas source and the gas coupler is configured to occur before an inlet of the gas compressor or an inlet of the gas pump, with the additional gas source comprising a valve to control the flow rate of the additional gas entering the gas coupler, thereby enabling regulation of the suction pressure at the inlet of the aspirator.

In some example implementations of the system, the gas coupler includes a component selected from the group consisting of a gas compressor and a gas pump, and wherein the fluid communication between the additional gas source and the gas coupler is established after an outlet of the gas compressor or an outlet of the gas pump, with the introduction of the additional gas into the nebulizing gas conduit serving to increase the overall gas flow rate within said conduit.

In some example implementations of the system, the system is configured such that the additional gas introduced into the nebulizing gas conduit is configured to advantageously enhance the disintegration of aerosol particles during the nebulization process and/or to facilitate the advantageous ionization of aerosol molecular species following the nebulization.

In some example implementations of the system, the system further comprises a filter, the filter being incorporated within the gas coupler and configured to limit the size of aerosol particles that reach the nebulizing gas conduit outlet.

In some example implementations of the system, the filter integrated within the gas coupler is a cyclone filter.

In some example implementations of the system, the system further comprises a check valve, the check valve being integrated within the gas coupler and configured to be open only when the gas within the gas coupler flows in the direction towards the nebulizing gas conduit outlet.

In some example implementations of the system, the check valve integrated within the gas coupler is a Tesla valve.

In some example implementations of the system, the system further comprises a hard obstacle positioned within the gas coupler, the hard obstacle being configured to induce turbulence in the mixture of nebulizing gas and aerosol particles as they pass around the obstacle, the turbulence contributing to at least partial disintegration of the aerosol particles within the mixture.

In some example implementations of the system, the system further comprises a hard obstacle situated within the gas coupler and positioned in the path of the aerosol particles traversing through the gas coupler, whereupon collision of the aerosol particles with this obstacle contributes to at least partial disintegration of the aerosol particles.

In some example implementations of the system, the liquid source unit comprises a liquid reservoir and a liquid pump, with the liquid pump being configured to transfer liquid from the liquid reservoir into the liquid conduit.

In some example implementations of the system, the liquid pump within the liquid source unit is a syringe pump.

In some example implementations of the system, the liquid conduit outlet and the nebulizing gas conduit outlet are integrated within a nebulizer device of a type selected from the group consisting of: cross flow nebulizer, V-Groove nebulizer, thin film nebulizer, parallel path nebulizer, enhanced parallel path nebulizer, Hildebrand grid nebulizer, and flow blurring nebulizer.

In some example implementations of the system, the liquid conduit and the nebulizing gas conduit are arranged concentrically relative to each other in the vicinity of the liquid conduit outlet or the nebulizing gas conduit outlet.

In some example implementations of the system, the liquid conduit resides at least partially within the nebulizing gas conduit.

In some example implementations of the system, the nebulizing gas conduit resides at least partially within the liquid conduit.

In some example implementations of the system, the system further comprises a source of secondary gas is connected to a secondary gas conduit, the secondary gas conduit comprising a secondary gas conduit outlet positioned in close proximity to the liquid conduit outlet.

In some example implementations of the system, the secondary gas is configured to assist in the nebulization process of the liquid.

In some example implementations of the system, the system further comprises a heater to pre-heat the secondary gas, such that upon mixing of the pre-heated secondary gas with the liquid droplets resulting from the nebulization, the pre-heated secondary gas contributes to the accelerated evaporation of these droplets.

In some example implementations of the system, the system further comprises a spray conduit, the spray conduit comprising a spray conduit inlet and a spray conduit outlet, the spray conduit being configured such that the spray conduit inlet encompasses a majority of the volume where nebulization occurs, and such that the stream of nebulizing gas propels the mixture of liquid droplets and aerosol material towards the spray conduit outlet.

In some example implementations of the system, the system further comprises an infrared radiation source positioned to heat the liquid droplets produced by the nebulization, thereby contributing to their accelerated evaporation.

In some example implementations of the system, the system is configured such that at least some aerosol molecular species are produced as a consequence of the nebulization process.

In some example implementations of the system, the system further comprises an ionization source configured to ionize the aerosol molecular species produced as a consequence of the nebulization process.

In some example implementations of the system, the ionization source is an atmospheric pressure ionization source.

In some example implementations of the system, the ionization method employed by the atmospheric pressure ionization source is selected from the group consisting of: electrospray ionization (ESI), atmospheric pressure chemical ionization (APCI), atmospheric pressure photoionization (APPI), and direct photoionization.

In some example implementations of the system, the ionization source comprises a conductor electrode at a high electric potential, positioned in close proximity to the liquid within the liquid conduit, such that the liquid volume exiting the conduit becomes charged, leading to the aerosol molecular species being charged either by acquiring some of the charges of the liquid during nebulization, or by electrospray ionization if the molecular species are entrapped in the charged liquid droplets resulting from the nebulization process, or by both of these two processes.

In some example implementations of the system, the system is configured such that the aerosol particle concentration at the nebulizing gas conduit outlet is time-dependent, wherein the high electric potential applied to the conductor electrode is pulsed, with such pulsing being synchronized with the temporal arrival of the aerosol particles at the outlet to optimize the charging process.

In some example implementations of the system, the system further comprises a micro-structured conductor that is in electrical contact with the conductor electrode and positioned such that the liquid is in close contact with the micro-structured features of the micro-structured conductor immediately before nebulization, whereby these micro-structured features increase exposure of the liquid to strong electric fields and consequently facilitate more efficient charging of the liquid exposed to nebulization.

In some example implementations of the system, the ionization source is an atmospheric pressure chemical ionization (APCI) system comprising an auxiliary corona discharge source, through which some of the generated aerosol molecular species transit, thereby acquiring charge via interaction with the corona discharge region.

In some example implementations of the system, the ionization source is an atmospheric pressure photoionization (APPI) system comprising a donor substance that mixes with the aerosol molecular species either during, after, or both during and after the nebulization process, and wherein the APPI system further comprises a UV light source that ionizes the molecules of the donor substance, which in turn transfer their charges to the aerosol molecular species, thereby achieving the ionization of these species.

In some example implementations of the system, the donor substance comprises a gas generated from a donor liquid component introduced into the liquid conduit, which evaporates either during the nebulization process, after it, or at both stages, thereby facilitating the ionization process.

In some example implementations of the system, the APPI system is configured such that the donor gas is introduced as a component of the nebulizing gas mixture through the nebulizing gas conduit outlet, thereby becoming an integral part of the nebulization process.

In some example implementations of the system, the ionization source is an optical source configured to emit radiation that is capable of directly ionizing the aerosol molecular species.

In some example implementations of the system, the system further comprises an ion funnel positioned to intercept the generated aerosol ions, such that the electro-magnetic fields of the ion funnel effectively guide a substantial portion of the aerosol ions towards an exit aperture of the ion funnel, thereby enhancing ion collection efficiency.

In some example implementations of the system, the system further comprises an ion transport conduit, the ion transport conduit comprising an ion transport conduit inlet and an ion transport conduit outlet, where the ion transport conduit inlet is positioned to capture ions emerging from the exit aperture of the ion funnel, facilitating the transfer of the ions towards the ion transport conduit outlet, which is directly connected to the inlet of a mass spectrometer, thereby enabling the efficient injection and subsequent analysis of the aerosol ions.

In some example implementations of the system, the system further comprises a gas funnel positioned such that the aerosol material, generated via the nebulization process, is efficiently guided into the gas funnel by the momentum of the nebulizing gas jet, ensuring that a substantial quantity of these particles is effectively directed towards an exit aperture of the gas funnel for subsequent processing or analysis.

In some example implementations of the system, the system further comprises a transport conduit having a transport conduit inlet and a transport conduit outlet, where the transport conduit inlet is positioned to receive aerosol particles emerging from the exit aperture of the gas funnel, the transport conduit being configured to efficiently transfer these aerosol particles towards the transport conduit outlet, the transport conduit being positioning to facilitate subsequent analysis of the aerosol particles discharged from the gas funnel.

In some example implementations of the system, the system further comprises an optical source, wherein the transport conduit comprises an optically transparent section, and wherein the optical source is positioned so that its emitted optical radiation passes through the optically transparent section, facilitating optical interaction with the aerosol particle fragments and aerosol molecular species that result from aerosol disintegration as they move through the transport conduit.

In some example implementations of the system, the system further comprises an optical detector positioned within or adjacent to the transport conduit, the optical detector being configured to detect at least a portion of the optical radiation following its interaction with aerosol particle fragments and aerosol molecular species.

In some example implementations of the system, the system is further configured such that the interaction between the optical radiation from the optical source and the aerosol particle fragments and aerosol molecular species encompasses one or more of the following processes: direct photoionization, optical excitation, or optical scattering.

In some example implementations of the system, the system is located such that nebulization occurs within an ambient gas environment, wherein said ambient gas is at or near atmospheric pressure.

In some example implementations of the system, the system is configured such that the ambient gas is selected from the group consisting of air, nitrogen, argon, helium, xenon, carbon dioxide, and combinations thereof.

In some example implementations of the system, the pneumatic nebulizer is configured such that the nebulizing gas is selected from the group consisting of air, nitrogen, argon, helium, xenon, carbon dioxide, and combinations thereof.

In some example implementations of the system, the pneumatic nebulizer is configured such that a pressure of the nebulizing gas within the nebulizing gas conduit ranges between 1 and 50 bars.

In some example implementations of the system, the pneumatic nebulizer is configured such that the volume flow rate of the nebulizing gas within the nebulizing gas conduit is within the range of 0.1 to 30 liters per minute.

In some example implementations of the system, the liquid source unit is configured such that a liquid flow rate within the liquid conduit is maintained within the range of 1 to 3000 μL/min.

In some example implementations of the system, the liquid solvent is selected from the group consisting of water, organic solvent, alcohol, methanol, ethanol, isopropanol, acetone, acetonitrile, dimethyl sulfoxide, chloroform, benzene, hexane, ethyl acetate, toluene, ionic liquid, and their mixtures.

In some example implementations of the system, the liquid source unit is configured such that the liquid emanating from the liquid source contains a chemical agent that advantageously reacts with at least a portion aerosol particle fragments and aerosol molecular species either during the nebulization process, within the liquid droplets resulting from the nebulization, or in both states.

In some example implementations of the system, the chemical agent comprises a molecular label that preferentially attaches to a specific subset of the aerosol molecular species, thereby facilitating the analysis of that subset.

In some example implementations of the system, the molecular label is selected from the group consisting of: enzymes, biotin, Halo tags, SNAP tags, nanoparticles, optical fluorescent labels, Raman labels, luminescent labels, radioactive isotopes, gold particles, magnetic labels, quantum dots, metal ions, or a combination thereof.

In some aspects, a system is provided comprising a first sub-system and a second sub-system, each sub-system being configured as described above, wherein the first sub-system is positioned relative to the second sub-system such that the aerosol material outputted from the first sub-system is utilized as the input aerosol material for the second sub-system.

In some example implementations of the system, the system further comprises a laser system capable of generating sample aerosol particles through laser ablation of a sample, wherein the aerosol particles are ejected from the sample as a plume.

In some example implementations of the system, the laser system operates at a wavelength or plurality of wavelengths ranging from 100 nm to 12 μm.

In some example implementations of the system, the laser output of the laser system is pulsed, with the duration of each laser pulse ranging between 10 femtoseconds and 1 millisecond.

In some example implementations of the system, the laser system is a component of a surgical device.

In some example implementations of the system, the laser system is a component of a tissue imaging device.

In some example implementations of the system, the system further comprises an imaging device configured such that prior to laser ablation, the sample is imaged using the imaging device to provide data that is employed for the selection of the laser sampling spot or to furnish additional information about the sample, augmenting the analysis of the sample aerosol particles.

In some example implementations of the system, the imaging device is a vibrational imaging device.

In some example implementations of the system, the vibrational imaging device employs an imaging modality is selected from the group consisting of Raman imaging, stimulated Raman scattering (SRS) imaging, coherent anti-Stokes Raman scattering (CARS) imaging, infrared imaging, and combinations thereof.

In some example implementations of the system, the system further comprises a photoacoustic imaging device configured such that prior to laser ablation, the sample is imaged using the photoacoustic imaging device to provide data that guides the determination of the laser sampling spot location or to furnish additional information about the sample, supplementing the analysis of the sample aerosol particles.

In some example implementations of the system, the system further comprises an optical coherence tomography (OCT) device configured such that prior to laser ablation, the sample is imaged using the optical coherence tomography (OCT) device to provide data that assists in determining the location of the laser sampling spot or to offer additional information about the sample, complementing the analysis of the sample aerosol particles.

In some example implementations of the system, the system further comprises an electrosurgical device configured to generate the sample aerosol particles via contact with a biological tissue.

In some example implementations of the system, the system further comprises a mass spectrometer positioned to receive ions generated from the aerosol particles, enabling the mass spectrometer to analyze the ions and thereby provide information about the chemical composition of the sample aerosol particles collected by the aspirator device.

In some example implementations of the system, the mass spectrometer includes an ion drift tube filled with a buffer gas, the ion drift tube comprising an ion drift tube inlet and an ion drift tube outlet, an ion trap in ion communication with the ion drift tube inlet, and an ion gating mechanism located at the ion drift tube outlet to regulate ion exit, wherein the ion trap accumulates aerosol ions and periodically releases them into the ion drift tube, and wherein the ion transition time through the ion drift tube is dependent on ion gas mobility within the buffer gas; and wherein the mass spectrometer measures the mass-to-charge ratio of ions exiting the ion drift tube.

In another aspect, there is provided a method for analyzing aerosol particles, the method comprising: employing the system as described above to obtain aerosol particles that have been at least partially disintegrated; and employing an analytic system to detect and analyze the at least partially disintegrated aerosol particles.

In some example implementations of the method, the analytic system comprises a mass spectrometer.

In some example implementations of the method, the aerosol particles are collected by the aspirator device, and wherein the at least partially disintegrated aerosol particles comprise ions, and wherein these ions are subsequently analyzed by the mass spectrometer.

In some example implementations of the method, the diameters of at least some of the sample aerosol particles are within the range of 1 nanometer to 100 micrometers.

In some example implementations of the method, the sample aerosol particles are generated from in vivo biological tissue.

In some example implementations of the method, the sample aerosol particles are generated from the ex vivo biological tissue.

A further understanding of the functional and advantageous aspects of the disclosure can be realized by reference to the following detailed description and drawings.

### BRIEF DESCRIPTION OF THE DRAWINGS

Embodiments are described with reference to the accompanying drawings. In the drawings, like reference numbers can indicate identical or functionally similar elements.

FIG. 1 shows a system for disintegrating aerosol particles, featuring an aspirator conduit drawing in particles, a gas coupler for fluid transfer, a nebulizing gas conduit with high-pressure gas for mixing, and a liquid source for collision-induced nebulization and particle disintegration near the outlets.

FIG. 2 illustrates a scenario where a pressure booster is integrated into the system to elevate the gas pressure inside the nebulizer gas conduit.

FIG. 3 shows the layout of embodiments with a passive pressure booster, featuring a gas source supplying motive gas through a conduit to the pressure booster, with options for the source being a pressure vessel or a gas compressor.

FIG. 4 illustrates the design of a passive pressure booster where motive gas flows through a constricted section of a tee connector, drawing in aerosol particles via Bernoulli's effect, resulting in a mixed stream exiting into the gas coupler outlet.

FIG. 5 illustrates an alternative design for a passive pressure booster using the Venturi effect, where motive gas and aerosol particles are drawn together at high velocity through constricted outlets, resulting in their mixture being discharged into the gas coupler outlet.

FIG. 6 depicts a system with two pressure boosters in series within the gas coupler for pressure enhancement between the aspirator inlet and nebulizing gas conduit outlet.

FIG. 7 shows an alternative embodiment where a supplementary gas source, either a pressure vessel or compressor, is integrated in parallel with the gas coupler's pressure booster through an additional gas coupler to enhance the flow rate of the nebulizing gas at the nebulizer conduit's outlet.

FIG. 8 presents a design for the additional gas coupler introduced in FIG. 7, where the supplementary gas conduit integrates with the principal gas coupler's outlet, featuring a concentric arrangement for efficient aerosol particle incorporation and pressure alignment.

FIG. 9 illustrates the enhancement of aerosol particle disintegration in a gas coupler through the placement of a rigid surface within the coupler, where high-velocity particles collide and partially disintegrate at the terminal end of a conduit.

FIG. 10 depicts an embodiment with an obstacle or constriction in the gas coupler, altering the gas and aerosol particle flow to create high Reynolds number flow and turbulent currents, leading to the fragmentation of softer particles for pre-nebulization disintegration.

FIG. 11a illustrates a linear aspirator conduit design, offering broad versatility for sampling aerosols across a sizable spatial domain.

FIG. 11b presents an aspirator conduit with an enlarged tip for targeted sampling in specific locations where aerosol particles concentrate.

FIG. 11c depicts an aspirator inlet divided into multiple sub-inlets merging into the main conduit, optimal for efficient capture in scenarios with limited aerosol production.

FIG. 12 illustrates an embodiment with an additional gas source, either a pressure vessel or compressor, integrated to allow customization of aspirator suction pressure, featuring a valve-regulated intermediary conduit for precise control and optimization of the aspirator's performance.

FIG. 13 shows an exemplary design for the secondary gas coupler from FIG. 12, where the intermediary conduit outlet integrates with the aspirator conduit inlet in a concentric arrangement, ensuring efficient incorporation of aerosol particles into the gas stream.

FIG. 14 illustrates the configuration of the liquid source in the system, comprising two primary components: a liquid reservoir and a liquid pump, for precise delivery of liquid substance to the liquid conduit.

FIG. 15a illustrates a specific embodiment using a concentric nebulizer, where the nebulizing gas conduit is concentric to, and encases, the liquid conduit within a housing unit.

FIG. 15b depicts the frontal view of the nebulizer illustrated in FIG. 15a, emphasizing the concentric arrangement with the liquid conduit inside the nebulizing gas conduit, clarifying the spatial relationships among the nebulizer elements.

FIG. 16a presents an alternative concentric nebulizer design where the nebulizing gas capillary is inside the liquid capillary, with an additional housing unit encapsulating both, enhancing aerosol particle disintegration by maximizing collisional impact with the liquid.

FIG. 16b provides a frontal view of this nebulizer configuration, detailing the spatial interplay among components, showing the gas capillary within the liquid capillary for improved dissolution efficiency of aerosol material.

FIG. 17a demonstrates a nebulizer design with a positive offset between the liquid and nebulizing gas capillaries, influencing nebulization efficiency, particle-liquid interaction, and ionization efficacy.

FIG. 17b displays a nebulizer configuration with a neutral offset, balancing efficiency, aerosol-liquid interaction intensity, and ionization effectiveness for aerosol molecular species.

FIG. 17c depicts a nebulizer design with a negative offset, where the nebulizing gas conduit outlet is recessed within the liquid capillary, impacting overall nebulizer efficiency and the interaction between aerosol particles and the liquid.

FIG. 18a shows an embodiment with a hot gas modification, where dry nitrogen is introduced via an external concentric conduit around the liquid conduit, merging into the nebulizing gas jet near the nebulization region, accelerating droplet evaporation.

FIG. 18b presents a frontal view of the nebulizer from FIG. 18a, emphasizing the spatial relationship and interaction between the hot gas, nebulizing gas, and liquid within the assembly for enhanced visual comprehension of the system's dynamics.

FIG. 19 illustrates the generic configuration of the hot gas conduit delivering hot gas to the nebulization region, showcasing that different geometrical arrangements are possible relative to the nebulizing area.

FIG. 20 depicts the use of an infrared lamp for heating liquid droplets to facilitate accelerated evaporation, highlighting the alignment of the lamp's infrared wavelength with the liquid's optical absorption peaks to enhance heating efficiency.

FIG. 21a introduces a ‘spray conduit’ designed to direct expanded nebulizing gas and aerosol material towards its outlet, confining the aerosol material within the conduit and enhancing analyte retention.

FIG. 21b depicts a spray conduit embodiment with an expanded cross-sectional outlet, engineered to facilitate laminar flow of the gas carrying the aerosol material for stable aerosol integration into analytical instruments.

FIG. 21c shows a design of a spray conduit with a concentric hot gas conduit, featuring perforations at the spray conduit to allow the hot gas to permeate the interior of the spray conduit and thus enhance evaporation of the spray droplets and prevent droplet accumulation on the inner walls of the spray conduit.

FIG. 22 shows the integration of ESI ionization in the design, featuring an electrical conductor in contact with the liquid in the liquid conduit and a high voltage supply, leading to ionization of the aerosol molecular species that are generated because of the nebulization.

FIG. 23 depicts an improved design of the liquid conduit outlet in a concentric nebulizer, featuring a stainless-steel inner capillary at high electric potential with micromachined channels to expand the surface area for electrochemistry, thereby enhancing liquid charging and ionization efficiency of aerosol molecular species.

FIG. 24 presents a solution for interfacing a grounded gas coupler with a high-voltage nebulizer gas conduit, featuring a grounded conductor through a ceramic interface, ensuring a gap larger than the dielectric breakdown distance for safe operation and effective aerosol particle injection.

FIG. 25 illustrates the integration of APCI into the design, featuring a corona discharge needle connected to a high voltage supply, positioned for ionizing aerosol molecular species as they pass through the corona region.

FIG. 26 shows a simplified embodiment for APPI integration, where a donor gas mixed with aerosol molecular species is exposed to UV light from a UV lamp, ionizing the donor gas which then transfers its charge to the aerosol species, leading to their ionization.

FIG. 27 depicts the integration of an ion funnel into the system, effectively directing aerosol ions formed during nebulization through its exit aperture and into an ion guiding conduit, significantly enhancing ion collection efficiency for mass spectrometry under atmospheric conditions.

FIG. 28 illustrates the use of a simple gas funnel for collecting neutral aerosol particle fragments and aerosol molecular species produced during nebulization, where the funnel, with its collection cone and gas flow, directs the aerosol particle fragments and aerosol molecular species towards its aperture and through a conduit for analysis.

FIG. 29 presents an optical excitation setup for aerosol particles in a conduit, featuring optically transparent windows for the passage of radiation from an external source and signal capture by an external apparatus, facilitating detailed optical analysis.

FIG. 30 demonstrates a typical setup for analyzing aerosols from laser plumes, where a sample undergoes laser ablation, creating a plume from which aerosol particles are captured and transported to an analysis system, as described in this invention, for processing into ions for mass spectrometry.

FIG. 31 illustrates a variation of the setup in FIG. 30, featuring a sample mounted on a motorized translation stage for multi-point sampling, allowing for the analysis of laser plumes at different locations and determination of the spatial distribution of molecular species within the sample.

FIG. 32 displays an alternate version of the setup in FIG. 30, where the laser light is directed to the sample via an optical fiber, with the fiber's output and aspirator inlet combined in a single, ergonomically designed housing unit suitable for handheld use.

FIG. 33 shows a scenario where an electrosurgical tool is used to aerosolize a biological tissue sample, with the resulting aerosol then analyzed using the system outlined in the context of FIG. 30.

FIG. 34 illustrates a system encompassing one or more embodiments of this invention generates ions from sample aerosol particles collected via an aspirator conduit, with ion generation occurring near the orifice of a mass spectrometer, which then collects and analyzes these ions.

### DETAILED DESCRIPTION

The following description and accompanying drawings provide an overview of various embodiments and aspects of this disclosure. These illustrations are intended to be exemplary rather than limiting, and the drawings are not necessarily drawn to scale. Detailed descriptions are included to ensure a comprehensive understanding of the embodiments. However, in some cases, commonly known or standard details may not be elaborated upon, to maintain a concise and focused presentation of the disclosure.

As used herein, the terms ‘incorporates,’ ‘comprises,’ and ‘comprising,’ along with their variations, are to be understood as inclusive and open-ended. They indicate that the specified features, steps, or components are included, but do not exclude the potential inclusion of other features, steps, or components.

Furthermore, the articles ‘a,’ and ‘an,’ are used herein to denote ‘at least one’ or ‘one or more,’ unless specified otherwise.

Additionally, the term ‘and/or,’ when used between two terms A and B, implies that either A, B, or both A and B can be present in the given context.

In the context of this document, the terms ‘including’ and ‘having’ are synonymous with ‘comprising.’

The term ‘exemplary,’ as used herein, denotes ‘serving as an example, instance, or illustration.’ It is noted that this term does not imply preference or superiority over other configurations described in this disclosure.

Additionally, ‘typical’ and ‘typically,’ when referring to an element, system, or behavior within an embodiment, indicate that such an element, system, or behavior is commonly present in various cases of the embodiment. However, these terms do not imply that these elements, systems, or behaviors are preferable or advantageous compared to other configurations presented herein.

In this disclosure, the terms ‘about’ and ‘approximately,’ particularly when associated with ranges of particle dimensions, mixture compositions, or other physical properties and characteristics, are intended to accommodate minor variations in the upper and lower limits of these ranges. This usage ensures that embodiments, which generally meet these dimensions on average but may include statistical outliers, are not excluded from the scope of this disclosure.

In this document, ‘disintegration’ is defined as ‘at least partial disintegration,’ and similarly, ‘disintegrate’ is to be understood as ‘to at least partially disintegrate,’ unless specified otherwise.

Furthermore, terms ‘gas compressor’ and ‘pump’ are used in their conventional senses as commonly understood in the field. A compressor refers to a mechanical device that increases gas pressure by reducing its volume. Conversely, a pump is defined as a device that moves a fluid, either a liquid or gas, from its inlet to its outlet, predominantly utilized to facilitate fluid movement through various conduits.

In this disclosure, ‘nebulization’ refers to the process of dividing a specific volume of liquid into smaller droplets, facilitated by the pneumatic action of a high-pressure gas.

Additionally, ‘outlet of a conduit’ or ‘conduit outlet’ encompasses all the outlets in a conduit with multiple outlets, unless specified differently.

Similarly, ‘inlet of a conduit’ or ‘conduit inlet’ encompasses all the inlets in a conduit with multiple inlets, unless specified differently.

In the context of this disclosure, characterizing aerosol particles encompasses a range of techniques and methods aimed at identifying and understanding the diverse properties and aspects of aerosol particles. This process may include, but is not limited to, any one or combination of the following: determining their physical, mechanical, and chemical properties; assessing their chemical composition and molecular structure; quantitatively measuring the concentrations or amounts of specific substances within the particles; and qualitatively determining the presence or absence of particular compounds within the particles. The specific characterization method(s) employed can be selected based on the particular requirements or objectives of the analysis, allowing for tailored investigation of the aerosol particles.

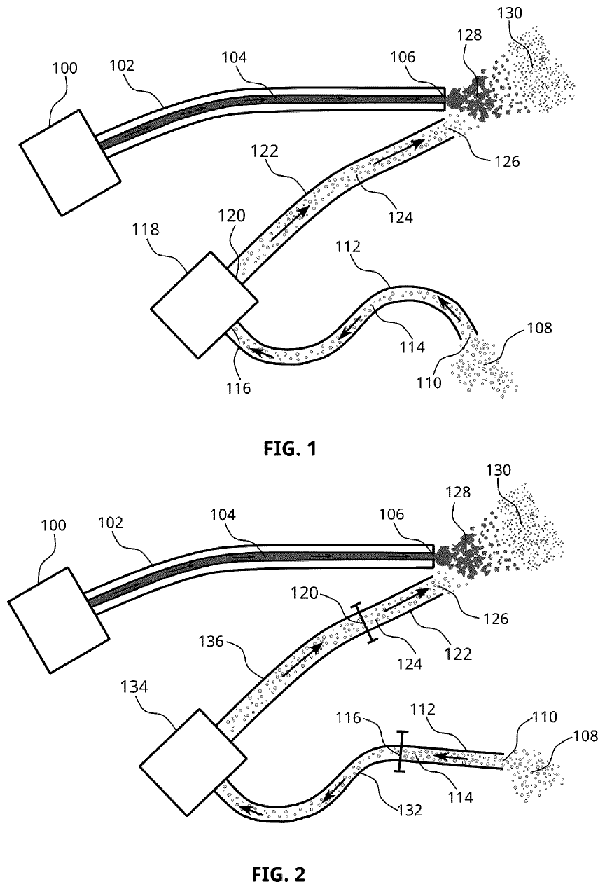

FIG. 1 illustrates a simple embodiment for a system for disintegration of sample aerosol particles. The process starts by drawing sample aerosol particles 108 into an aspirator conduit 112 through the aspirator conduit inlet 110. Within this conduit, the aerosol particles, identified as 114, are conveyed towards a gas coupler, indicated as 118. The gas coupler 118 features an interface, marked as 116, establishing fluid communication between the aspirator conduit 112 and the gas coupler 118. An additional interface, designated as 120, on the gas coupler 118 facilitates fluid communication between the gas coupler 118 and the entrance of a nebulizing gas conduit, referred to as 122, which is filled with high-pressure nebulizing gas. The primary function of the gas coupler 118 involves the introduction of aerosol particles 114 from the aspirator conduit 112 into the high-pressure nebulizing gas stream within the nebulizing gas conduit 122. The resultant mixture, comprising the aerosol particles and the high-pressure nebulizing gas, denoted as 124, is then discharged from the nebulizing gas conduit 122 via its outlet, referred to as 126.

In a separate aspect of the system, a liquid source, identified as 100, provides a liquid, marked as 104, to a liquid conduit, designated as 102. This liquid 104 is expelled from the liquid conduit 102 through its outlet, labeled as 106.

The outlet 106 of the liquid conduit 102 is strategically positioned proximate to the outlet 126 of the nebulizing gas conduit 122, enabling the high-pressure mixture 124 emanating from the outlet 126 to intersect with the liquid dispensed from the outlet 106. The interaction of the mixture 124 with the emitted liquid culminates in the nebulization of the liquid within a designated nebulization space, indicated as 128, in the proximity of outlets 106 and 126. This collision significantly contributes to the partial disintegration of the aerosol particles, which represents a key objective of the present disclosure.

Moreover, in instances where the liquid functions as a solvent and the aerosol material is soluble, a portion of the aerosol material may dissolve in the liquid during their interaction in the nebulization process. Additionally, some aerosol material may become entrapped and partially dissolved within the liquid droplets emanating from the nebulization space 128. Overall, this dissolution process can contribute to the desired disintegration of the aerosol particles.

The nebulization process is typically enhanced after the primary collision through the turbulence of the nebulizing gas within the nebulization space 128 which causes formed droplets to collide with each other and with the aerosol material. These secondary collisions can contribute to both breaking droplets into even smaller droplets and to additional disintegration of the aerosol material.

The efficiency of the aerosol disintegration process can be optimized by optimizing various parameters of the nebulization process which include, but are not limited to, the specific nature of the liquid utilized, its flow rate, the type of gas employed for nebulization, alongside its flow rate and pressure.

Additionally, the design features of the outlets, referenced as 106 and 126 in the FIG. 1, are relevant. Particularly, the strategic positioning of outlet 126 in relation to outlet 106 is of essence. An exemplary configuration is such that the high-velocity gas emanating from outlet 126 induces a low-pressure zone adjacent to the liquid outlet 106, pursuant to the Bernoulli's principle. This phenomenon effectively facilitates the entrainment of the liquid into the gas stream, thereby promoting an interactive collision between the liquid and the aerosol material. Furthermore, the efficiency of the nebulization process is further augmented by restricting the extent of the nebulization space 128. Such confinement serves to maintain elevated levels of gas pressure and turbulence for a prolonged duration, simultaneously ensuring that both the liquid droplets and the aerosol material undergo a higher frequency of collisions within the constrained space.

The general foundational principles of nebulization processes are well known to a person skilled in the art. This knowledge permits the adaptation of a multitude of existing nebulizer designs to integrate specific features such as the outlets 106 and 126, characterized by their geometrical configurations, material compositions, and respective positional relationships. Additionally, these designs may incorporate a designated chamber to precisely define the boundaries of the nebulization space, denoted as region 128. Certain designs of nebulizers may exhibit preferential compatibility with certain applications. The process of identifying the most appropriate nebulizer design and fine-tuning the operational parameters of the nebulization process can be effectively guided by employing a range of diagnostic techniques familiar to those versed in the field. These techniques include laser diffraction spectroscopy, phase Doppler particle analysis, and high-speed imaging methodologies. Furthermore, the examination of properties of the aerosol material post-nebulization serves as a tool for both the characterization and enhancement of the nebulization process.

As used herein, the term “aerosol transport” means transporting aerosol particles from the aspirator inlet 110 to the outlet 126 of the nebulizer conduit 122.

As used herein, the term “aerosol collection” means collecting the aerosol particle fragments and aerosol molecular species created during and after the nebulization event and directing these aerosol particle fragments and aerosol molecular species toward the analytical device where the analysis of these aerosol particle fragments and aerosol molecular species takes place.

The liquid source 100 may contain various elements needed for supplying a proper liquid with a proper flow rate to the liquid conduit. The liquid source may comprise one or several liquid reservoirs, one or several liquid pumps, liquid mixers, liquid heaters, various liquid conduits, and various liquid couplers.

On some occasions, the sample aerosol particles can be initially present within a gas environment that has a pressure large enough to push the aerosol particles inside the aspirator, move the aerosol particles toward the nebulizer conduit outlet 126, and cause the nebulization without any active elements within the gas coupler. In this case, the gas coupler can be just a simple tube adapter or connector.

However, in many cases of interest it can be beneficial to boost the gas pressure at the outlet of the nebulizing gas conduit relative to the gas pressure at the aspirator inlet to achieve a successful nebulization. The typical nebulizing gas pressures in pneumatic nebulizers are in the range of 2-10 bars while the sample aerosol particles are typically sampled from environments with pressures close to the standard atmospheric pressure of 1 bar.

This more realistic scenario is illustrated in FIG. 2. Here the gas coupler 118 from FIG. 1 is split into a pressure booster 134 that is connected to the interface 116 through a gas coupler inlet conduit 132 and to the interface 120 through a gas coupler outlet conduit 136. Other elements in FIG. 2 have the same annotations and meanings as those in FIG. 1.

The pressure booster can be any device for increasing pressure in a gas delivery system known to a person skilled in the art. Examples are a mechanical device such as a gas compressor or a passive device such as a Venturi pump. By their nature, pressure boosters ingest gas into their inlets so they naturally provide the suction pressure at the aspirator inlet in our case.

Among mechanical gas compressors, a scroll compressor could be a good choice because there are commercial medical-grade OEM models used for surgical smoke evacuation that already exist. These models are oil free and made with extreme tolerances to avoid gas leakages and noise. The scroll pumps may have an additional benefit of creating turbulence during the compression process which may aid to aerosol particle disintegration. In some other cases, embodiments with diaphragm compressors or some other mechanical gas compressor type could be more beneficial.

In some cases, a passive pressure booster could be advantageous for its simplicity and for reduced interaction between the pressure booster and the aerosol particles. Most known passive pressure boosters are based on the Venturi effect. Such devices create negative pressure by passing a motive gas through a constricted section of a pipe or tube, so the velocity of the motive gas increases and concurrently, its pressure decreases according to Bernoulli's principle.

This negative pressure can be used to draw the gas with the aerosol particles from the aspirator into the stream of the motive gas.

Since the pressure of the motive gas is typically high, the mixture of these gasses and the aerosol particles alone may enter the nebulizing gas conduit with sufficient kinetic energy to perform the nebulization.

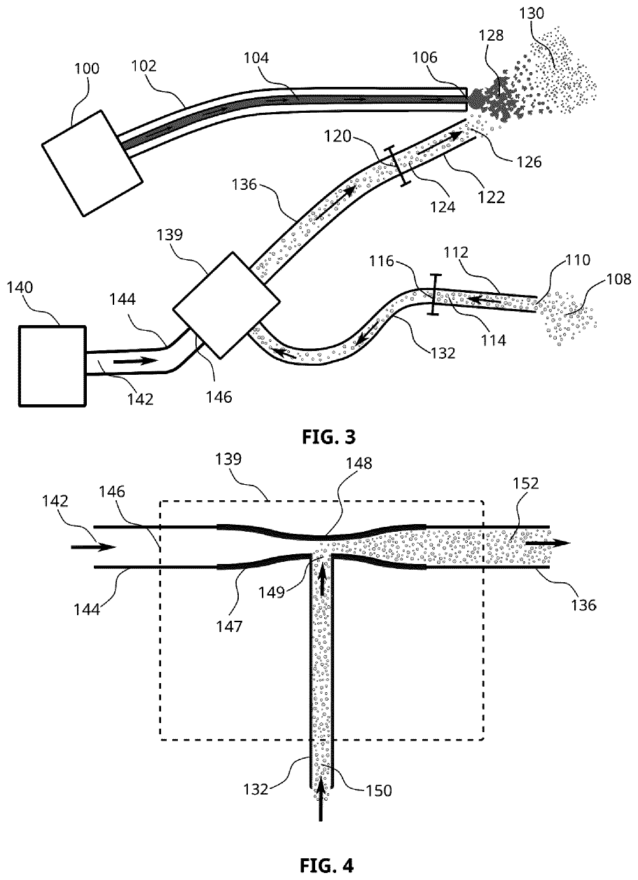

The general layout of embodiments with a passive pressure booster is shown in FIG. 3. The layout comprises a gas source 140 that supplies the motive gas 142 to the motive gas conduit 144 which is in fluid communication with the passive pressure booster 139 through the interface 146. The source 140 can be a pressure vessel or a gas compressor. Other elements in FIG. 3 have the same annotations and meanings as those in FIG. 2.

FIG. 4 shows a possible design for the passive pressure booster 139. After entering the pressure booster 139, the motive gas 142 flows through a constricted section 148 of a tee connector 147 where the constricted section also has another inlet 149 which is in fluid communication with the inlet 132 where the aerosol particles 150 are coming from. Due to the negative pressure at the constricted section 148 created through the Bernoulli's effect, the aerosol particles 150 get drawn into the stream of the motive gas 142 and the mixture of the motive gas and the aerosol particles 152 exit the pressure booster 139 into the gas coupler outlet 136.

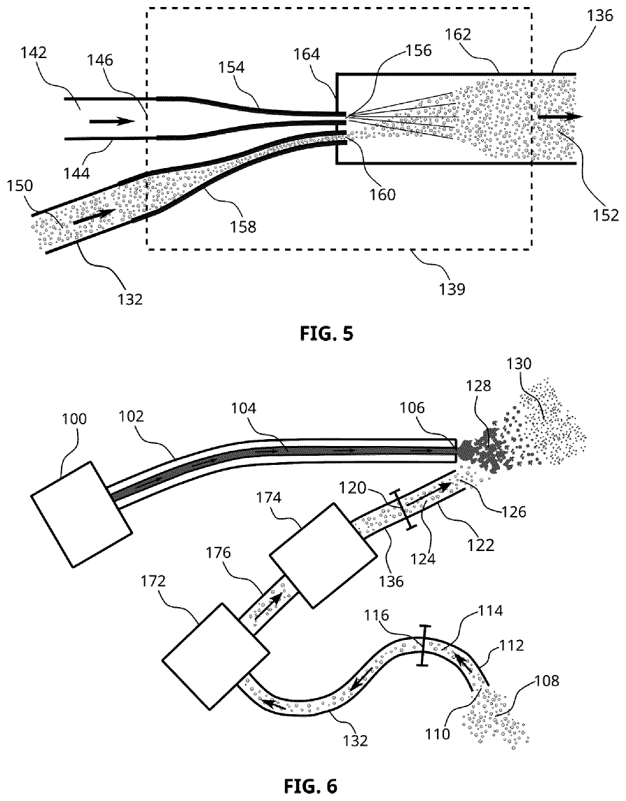

Another possible design for a passive pressure booster based on the Venturi effect is shown in FIG. 5. In this case the motive gas conduit 144 is coupled into the passive pressure booster 139 through a pipe 154 with a constricted outlet 156 where the motive gas exits with high velocity. The conduit 132 that carries the aerosol particles 150 is coupled to another pipe 158 whose outlet 160 is in vicinity of the outlet 156. Due to the Venturi effect in the vicinity of the outlet 156, the gas carrying the aerosol particles in the pipe 158 gets drawn into the stream of the motive gas exiting the outlet 156. Both outlets 156 and 160 are encompassed by a conduit 162 whose backend 164 is sealed. Finally, the mixture of the motive gas and aerosol particles 152 gets discharged into the gas coupler outlet 136.

If a large pressure jump is needed between the aspirator inlet and the outlet of the nebulizing gas conduit, it could be advantageous to add two pressure boosters in series within the gas coupler as shown in FIG. 6. In this case, the outlet of the first pressure booster 172 is in fluid communication 176 with the inlet of the second pressure booster 174. Other elements in FIG. 6 have the same annotations and meanings as those in FIG. 2. The pressure booster types can be of those discussed above. The design principles of adding two pressure boosters in series are known to a person skilled in the art.

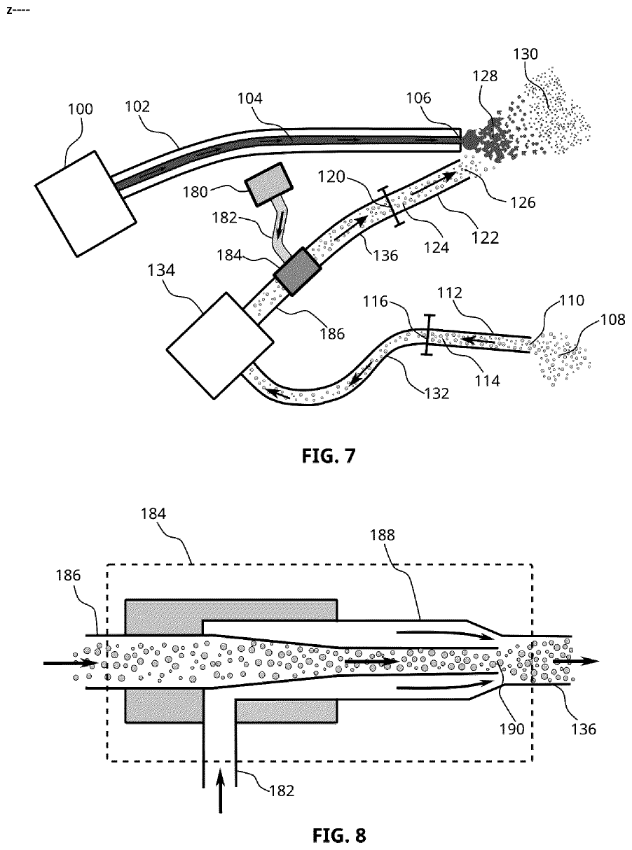

In an alternative embodiment to enhance the flow rate of the nebulizing gas at the exit point of the nebulizing gas conduit, a supplementary gas source is incorporated in a parallel configuration with the pressure booster of the gas coupler. Referencing FIG. 7, this concept is depicted, wherein a gas source, labeled as 180, which may be either a pressure vessel or a gas compressor, is designed to establish fluid communication with the outlet 136 of the gas coupler. This connection is facilitated through an additional gas conduit, identified as 182. The integration of the gas source into the system is achieved by an additional gas coupler, marked as 184. This arrangement enables the introduction of gas from the supplementary source 184 into the pre-existing gas stream originating from the pressure booster 134, thereby augmenting the gas flow rate at the discharge point of the nebulizer conduit. It should be noted that other elements presented in FIG. 7 retain the same designations and connotations as those elucidated in FIG. 2.

FIG. 8 delineates a feasible embodiment for the additional gas coupler 184, as previously introduced in FIG. 7. Within this additional gas coupler 184, the supplementary gas conduit 182 integrates seamlessly with the outlet 136 of the principal gas coupler. A distinctive feature of this design is the configuration of the terminal segment 190 of conduit 186, which links the output of the pressure booster to the input of the additional gas coupler. This terminal segment 190 is strategically positioned to be concentric with, and enclosed within, the outlet 136 of the main gas coupler. Such an arrangement ensures that the supplementary gas envelops the terminal 190 of conduit 186, thereby facilitating an efficient incorporation of aerosol particles emanating from conduit 186 into the stream within conduit 136. A design specification necessitates that the pressure of the supplementary gas surrounding the terminal of conduit 186 aligns closely with the internal gas pressure at the terminal 190 of conduit 186. It should be noted that the principles governing the design of gas sources in parallel configurations are commonly understood by those skilled in the relevant technical field.

The additional gas introduced as depicted in FIG. 7 may offer benefits extending beyond the mere augmentation of the flow rate within the nebulizing gas conduit. This gas plays an active role in the nebulization process and, due to its thorough mixing with the resultant aerosol material, can be specifically selected for its chemical composition to facilitate the analysis of the aerosol molecular species produced by the nebulization. For instance, the chosen gas can function as a charge donor during the atmospheric pressure photoionization (APPI) of the aerosol molecular species.

In further embodiments, additional components may be incorporated into the main gas coupler, not limited to pressure boosters, with the objective of enhancing aerosol analysis, simplifying maintenance, or augmenting the user experience.

One such example is the integration of a particle filter within the gas coupler. This filter is designed to selectively allow aerosol particles of a specific size to pass, effectively intercepting larger particles. The utilization of cyclone filter designs is particularly advantageous in this context. Their contactless sorting mechanism and the convenience they offer in the disposal of filtered materials make them highly suitable. Cyclone filters are generally configured for the control and elimination of particulate matter with diameters exceeding 10 micrometers, aligning with one of the envisaged application scenarios.

In a further embodiment of the present disclosure, the gas coupler may incorporate a check valve, particularly advantageous in scenarios where a significant pressure differential exists between the outlet of the nebulizing conduit and the inlet of the aspirator. This inclusion addresses potential complications arising from the obstruction of the gas pathway, where high-pressure gas might otherwise reverse its direction, leading back to the inlet of the aspirator, thereby causing operational disruptions. A preferred embodiment for this check valve is the utilization of a Tesla valve. Its elegantly simple, yet highly effective design is characterized by the absence of moving parts, thus enhancing reliability, and reducing maintenance requirements in the context of the present disclosure.

Other standard features known to a person skilled in the art could be added to the gas coupler for controlling and characterizing the aerosol flow through the gas coupler: vents, relief valves, flow meters, pressure gauges, etc.

In the present disclosure, while nebulization serves as the primary method for aerosol particle disintegration, it may be beneficial to initiate a preliminary phase of aerosol disintegration within the gas coupler, prior to the primary nebulization process. This preliminary disintegration phase is particularly advantageous in instances where the aerosol contains particles of larger dimensions. By facilitating an initial reduction in particle size, this pre-nebulization disintegration stage ensures the provision of smaller particles for the subsequent main nebulization event. Such a process is designed to enhance the overall efficiency of particle disintegration, resulting in a more uniform and effective final aerosol composition.

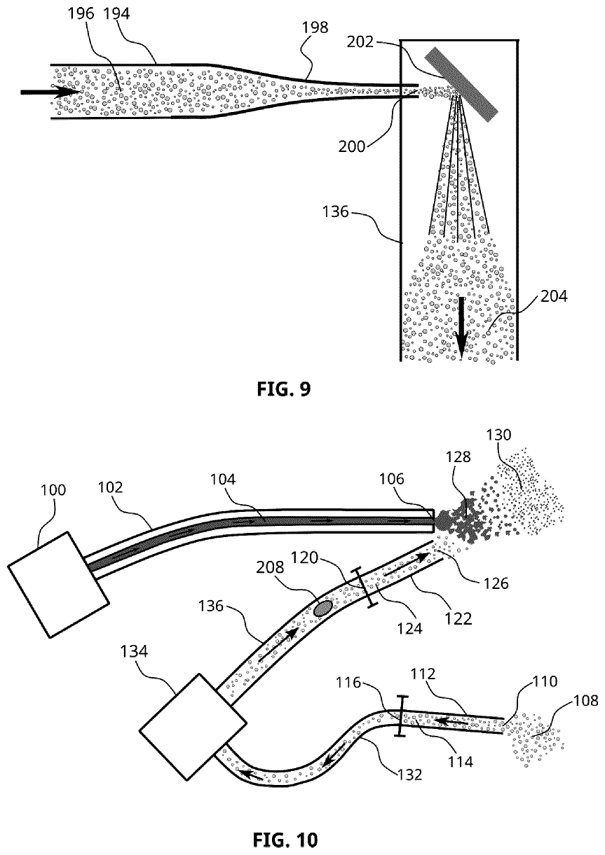

A straightforward approach for enhancing aerosol particle disintegration within the gas coupler involves the strategic placement of a rigid surface within the gas coupler, as delineated in FIG. 9. The aerosol particles 196, which, upon being propelled from the pressure booster, traverse through a conduit 194. These particles are then accelerated towards the constricted terminal end 198 of the conduit 194 with an outlet 200, which is aptly positioned within the outlet 136 of the gas coupler. In close proximity to the outlet 200, a rigid surface is strategically located within the outlet conduit 136. This surface acts as an interceptor for the high-velocity aerosol particles emanating from the outlet 200. The ensuing collisions between the particles and the rigid surface result in their partial disintegration. Subsequently, the fragmented aerosol material 204 continues its passage through the gas coupler outlet conduit 136.

Notwithstanding the foregoing, it should be noted that the collision of aerosol particles with rigid surfaces, as previously discussed, may present drawbacks, such as the adherence of particle residuals on the hard surfaces. This can result in a diminution of signal strength and potential signal contamination. To mitigate these issues, especially in scenarios where aerosol particles are of a softer constitution, an alternate embodiment for pre-nebulization disintegration is proposed and illustrated in FIG. 10. In this embodiment, an element such as an obstacle 208 or a constriction is strategically incorporated within the gas coupler. This configuration is designed to modify the path of the gas carrying the aerosol particles, either around the obstacle or through the constriction. Such an arrangement induces a flow characterized by a high Reynolds number, leading to the generation of turbulent currents. Provided that the turbulence is of sufficient intensity, it can effectively fragment some of the softer aerosol particles, thereby achieving the desired pre-nebulization disintegration. The roles and annotations of other elements in FIG. 10 are equivalent to those from FIG. 2.

In reference to FIG. 2, the aspirator conduit 112 is characterized as a passive component, primarily functioning to facilitate the ingress of sample aerosol particles into its inlet via suction, and subsequently transporting these particles to the gas coupler. Despite its passive nature and the absence of active elements within its structure, certain characteristics of the aspirator conduit—such as its material composition, geometric design, and the applied suction pressure—play a pivotal role in influencing the efficiency of aerosol sampling. Consequently, these factors bear a significant impact on the overall efficacy of the aerosol analysis process.

The construction of the aspirator conduit should employ materials that are chemically inert in relation to the aerosol particles. Typically, but not exclusively, materials such as stainless steel, Teflon, and PEEK (Polyether Ether Ketone) plastic are preferred for their inert properties in these applications. Additionally, regulating the temperature of the aspirator conduit may prove advantageous for minimizing interactions between the aerosol and the surface of the conduit. This can be achieved by positioning heating or cooling elements in close proximity to the conduit. An exemplary implementation of this strategy could involve maintaining the temperature of the conduit significantly above the dew point to preclude surface condensation.

The geometric configuration of the aspirator conduit plays a role in its functionality. This includes the design of the inlet of the aspirator conduit as well as the specific cross-sectional area of both the conduit and its inlet. Notably, a reduced cross-sectional area within the conduit contributes to an increased velocity of the gas passing through it. This acceleration aids in mitigating the adhesion of aerosol particles to the internal surfaces of the conduit, as the rapid gas flow facilitates their removal. Conversely, an excessively diminutive cross-sectional area may lead to recurrent obstructions within the conduit, necessitating more frequent maintenance. Additionally, in certain instances, the gas velocity at the inlet may become excessively high due to substantial suction pressure. In such scenarios, integrating vents along the conduit wall can be beneficial. These vents are designed to draw in additional gas, thereby moderating the velocity of the gas at the inlet.

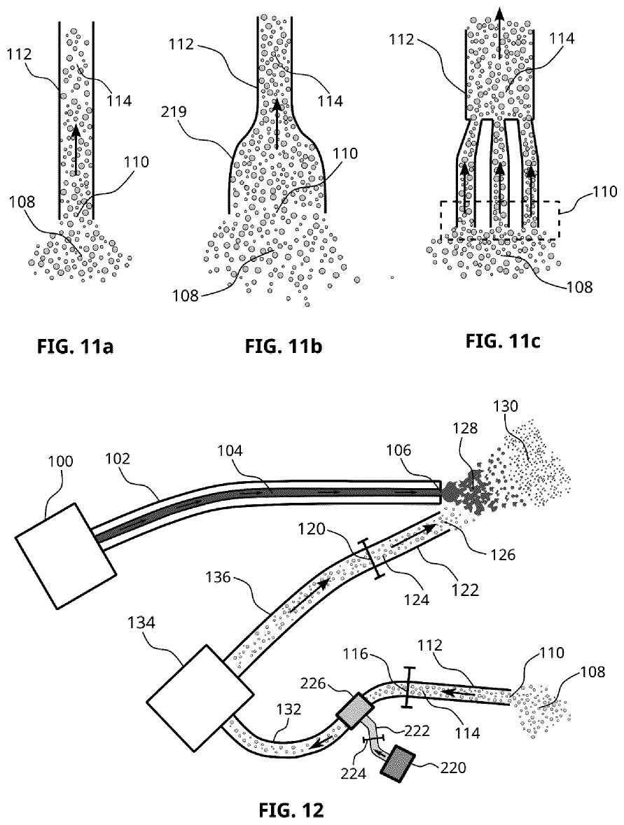

In FIG. 11, various illustrative but non-limiting geometrical configurations for the aspirator conduit and its inlet are presented. In FIG. 11a, we observe a straightforward linear aspirator conduit design, exhibiting broad versatility due to its capacity to sample a sizable spatial domain effectively. Moving on to FIG. 11b, we encounter an exemplary implementation of the aspirator conduit tailored for sampling aerosols generated at specific locations. In this embodiment, the aspirator conduit features a tip 219 characterized by an enlarged cross-sectional area, strategically encompassing the volume in which most of aerosol particles tend to concentrate.

Lastly, in FIG. 11c, we explore an embodiment wherein the inlet 110 of the aspirator is divided into multiple sub-inlets, typically two or more, which subsequently merge into the primary conduit 112. This configuration holds promise, for example, in scenarios where a limited volume produces a relatively small quantity of aerosol particles. The positioning of the sub-inlets can be optimized to enhance the capture efficiency for as many aerosol particles as feasible, thus enhancing overall effectiveness.

In cases where aerosol particles are produced by a device (such as a laser), it could be advantageous to incorporate the aspirator conduit within an integrated apparatus that encompasses both the aspirator conduit and the said device.

Sampled aerosol particles typically originate from diverse environments, with a predominant occurrence in gaseous atmospheres approximating standard atmospheric pressure. In many instances, the optimization of the suction pressure of the aspirator to align with specific operational requisites proves advantageous. As elucidated in FIG. 12, a potential embodiment has been devised to facilitate this tailored customization.

Within this configuration, an additional gas source, denoted as 220, is seamlessly integrated into the system. This gas source can manifest as a pressure vessel or a gas compressor, thereby offering versatility in adaptation. Fluid connectivity is established between this gas source and the inlet conduit 132 of the gas coupler through the intermediary conduit 222 that is coupled into the inlet 132 through a secondary gas coupler 226. To regulate the quantity of gas released from the gas source into the inlet 132, a valve 224 is strategically positioned along the conduit 222.

It is worth noting that the gas flow rate within the inlet 132 results from the combined contributions of the gas drawn into the inlet of the aspirator and the supplementary gas introduced from the source 220. Primarily governed by the operation of the pressure booster 132, increasing the gas flow through the valve 224 inversely reduces the suction pressure at the inlet of the aspirator 110, and vice versa. This dynamic adjustment mechanism empowers users with precise control, facilitating the fine-tuning of the suction pressure of the aspirator to align with specific requirements, thereby optimizing its performance.

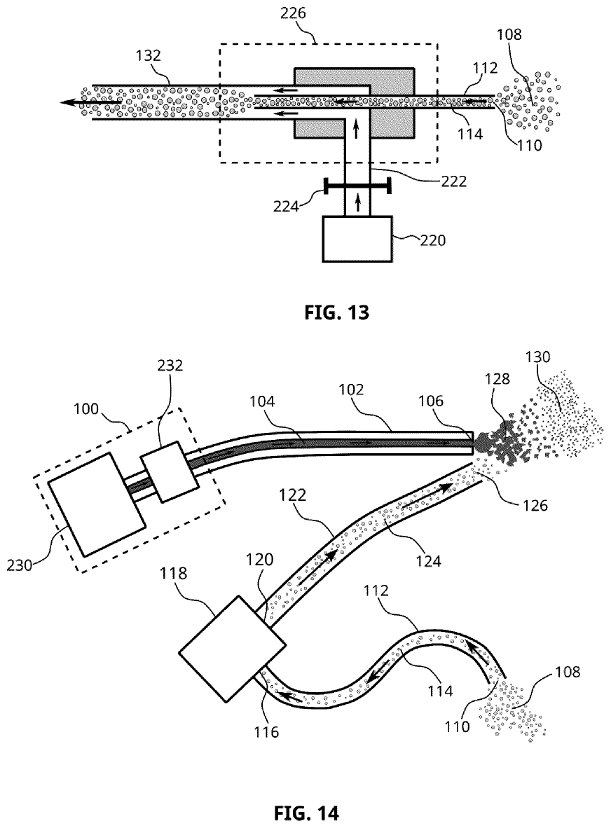

FIG. 13 presents an exemplary embodiment for the secondary gas coupler 226 from FIG. 12. The outlet of the intermediary conduit 222 integrates harmoniously with the inlet of conduit 132. The outlet of the aspirator conduit 114 is strategically positioned to be concentric with, and enclosed within, the outlet of the intermediary conduit 222. Such an arrangement ensures that the additional gas originated from the source 220 envelops the outlet of conduit 114, thereby facilitating an efficient incorporation of aerosol particles emanating from conduit 114 into the stream within conduit 132.

Beyond its role in controlling aspirator suction pressure, the utilization of the supplementary gas sourced from 220 offers a few other versatile applications within the gas coupler. For instance, the gas can be selectively heated to impart advantageous thermal treatment to the aerosol particles within the confines of the gas coupler. Furthermore, specific gases with advantageous chemical properties can be employed to interact with the aerosol particles within the gas coupler. Generally, however, dry nitrogen can be used for its inert properties.

To ensure the efficient conveyance of a substantial proportion of aerosol particles from the inlet of the aspirator to the outlet of the nebulizing conduit, it is imperative to minimize losses of aerosol particles within the transportation system.

This necessitates the careful consideration of each individual component, encompassing conduits, pumps, connectors, and the like, with a focus on reducing impediments to gas flow and minimizing dead volume. Moreover, maintaining an elevated temperature within the transportation system is essential to prevent the undesirable condensation of the gases in which the aerosol particles are suspended. Also, it is noted that the materials the transportation system is made of are chemically inert and do not chemically interact with the aerosol particles in undesired ways. The typical materials for building such a system typically include stainless steel, Teflon, and PEEK plastic.

A more nuanced yet significant concern revolves around the mitigation of static electricity within the transportation system. Given the lightweight and diminutive nature of aerosol particles, they are susceptible to being attracted and trapped by statically charged surfaces. Plastic tubing poses a notable challenge in this regard, as the flow of gas through such tubing can readily induce a static charge on the plastic material, resulting in the inadvertent attraction of aerosol particles within the gas stream.

Practitioners skilled in the relevant field have devised various strategies to address this issue. One commonly adopted approach involves employing tubing with inner walls that possess partial conductivity, enabling them to be grounded for the discharge of static electricity. In this context, an exemplary implementation for our embodiments entails the utilization of carbon-doped PTFE tubing. This specialized PTFE tubing incorporates a carbon dopant, typically in the range of 2-3%, rendering it sufficiently conductive to effectively dissipate charges from the inner conduit walls while preserving the chemical inertness characteristic of pure PTFE.

Another possible solution to counter the challenge of aerosol particle adhesion to surfaces due to static electricity involves the deployment of an ultrasonic transducer affixed to said surface. This transducer induces vibrations within the surface, effectively dislodging the particles that may have adhered due to static attraction.

The integral function of the liquid source within our system, identified as 100 in the previous discussions, is to facilitate the precise delivery of liquid substance 104 to the liquid conduit 102. As elucidated in FIG. 14, the typical configuration of this liquid source consists of two primary components: a liquid reservoir (referred to as 230) and a liquid pump (designated as 232).

While the choice of liquid pump may encompass various types familiar to those skilled in the field, there is a preference for pumps capable of dispensing small quantities of liquid with a high degree of precision. Syringe pumps exemplify such preferred devices, offering accuracy in the delivery of minute liquid volumes.

In more advanced applications, there is an opportunity to employ more intricate liquid sources that encompass multiple reservoirs, each containing distinct liquids. This multiplicity of liquids can be advantageously combined in predetermined proportions within a liquid mixer, with the resulting mixture subsequently being conveyed to the liquid conduit via a shared pump. This configuration provides the versatility to handle diverse liquid compositions, enhancing the adaptability and capabilities of the system for advanced applications.

Another valuable augmentation to the liquid source pertains to the inclusion of a liquid temperature controller, designed to regulate the temperature of the liquid prior to nebulization. This temperature control offers multiple advantages, including enhancing the stability of analytical signals. Furthermore, it provides the capability to elevate the temperature of the liquid. The application of elevated liquid temperatures can facilitate the disintegration of aerosol particles, potentially optimizing their behavior. Additionally, heightened temperatures can expedite the evaporation of the liquid droplets generated during the nebulization process.

The selection of the liquid used for the nebulization is thoughtfully determined by the characteristics of the aerosol particles in question and the specific analytical methodology employed for aerosol analysis. Frequently, the use of a solvent proves advantageous due to its ability to facilitate the partial or complete dissolution of aerosol particles and fragments. This dissolution may occur either during the nebulization process or subsequently within the liquid droplets produced as a result of nebulization.

A broad spectrum of solvents is available for use, contingent upon the unique requirements of the application. These solvents encompass organic solvents, water, various alcohols such as methanol, ethanol, isopropanol, and acetone, along with acetonitrile, dimethyl sulfoxide, chloroform, benzene, hexane, ethyl acetate, ionic liquids, or blends thereof.

An additional promising application of the liquid component lies in its capacity to enhance the ionization process of aerosol molecular species. One illustrative scenario involves the introduction of a specific quantity of toluene into the liquid medium. After the formation of a fine spray through the nebulization process and the subsequent evaporation of the spray droplets, the resulting aerosol molecular species can undergo ionization utilizing atmospheric pressure photoionization (APPI), wherein toluene serves as the charge donor.

A further advantageous application of the liquid component resides in Its potential utilization as a conduit for the delivery of molecular labels. These labels find extensive application in the realms of biological and medical research, serving the vital functions of detecting, pinpointing, and monitoring specific molecules or entities within biological systems. Exemplary molecular labels encompass a range of substances such as enzymes, biotin, Halo tags, SNAP tags, nanoparticles, optical fluorescent labels, luminescent labels, radioactive isotopes, metal isotopes, gold particles, metal magnetic labels, quantum dots, metal ions, or any synergistic combination thereof. Within the framework of the present disclosure, characterized by its expeditious intermingling of aerosolized molecular species with the liquid medium during nebulization, and within the resultant liquid droplets derived thereof, a unique avenue is afforded for the real-time labeling of aerosolized molecular species of interest. This capability is contingent upon the initial presence of said labels within the liquid medium subject to nebulization, thereby facilitating dynamic molecular labeling.

As previously mentioned, the present disclosure is compatible with various existing nebulizer designs, including but not limited to the cross flow, V-Groove, thin film, parallel path, enhanced parallel path, Hildebrand grid, and flow blurring nebulizers. In this application, several embodiments are presented that utilize concentric nebulizers, a nebulizer type extensively employed in analytical instrumentation.

In a concentric pneumatic nebulizer, the liquid conduit and the nebulizing gas conduit are concentric. The internal and outer diameters for these conduits are typically small enough that these conduits can be considered to be capillaries; so, we will refer to them as liquid and nebulizing gas capillaries. The inner tube, which is narrower, is typically used for transporting the liquid sample. The outer tube, which is wider, typically carries the nebulizing gas.

These nebulizer types are predominant in pneumatic electrospray ionization (ESI) sources utilized in mass spectrometry. Over the past 40 years, they have undergone extensive development, characterization, and testing. As a result, their fundamental design parameters, such as liquid and gas flow rates and the cross-sectional areas of the capillaries, are well-established and recognized by experts in the field. These parameters can serve as foundational guidelines for the initial design of other nebulizer embodiments in the present disclosure, which can subsequently be fine-tuned to achieve optimized performance.

The liquid flow rates in commercial pneumatic ESI devices in mass spectrometry are typically between 50 L/min and 1 mL/min and the inner diameters of liquid capillaries are typically in the 10-200 μm range. To nebulize this amount of liquid, nebulizing gas flow rates in the range of 0.1-5 L/min are used where the nebulizing gas conduit typically has the inner cross-sectional area that is equivalent to a capillary with the inner diameter in the 100-200 μm. The typical gauge pressures of the nebulizing gas source needed to achieve these flow rates are in the range of 20-60 PSI. The actual set of the parameters that are used mostly depends on the given sample flow rate (as in liquid chromatography) and the properties of the solvent (e.g. surface tension and viscosity).

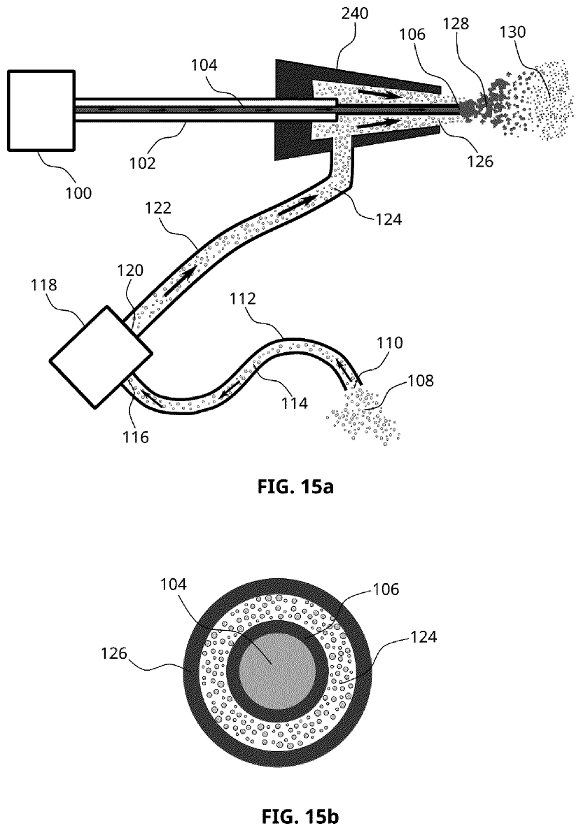

Referring to FIG. 15a, a specific embodiment employing a concentric nebulizer is depicted. This embodiment parallels the arrangement and functionality of the elements detailed in FIG. 1, with corresponding annotations for clarity. In this design, the terminal segment of the nebulizing gas conduits encases the terminal segment of the liquid conduit, both of which are seamlessly incorporated into housing unit 240. During operation, as the gas facilitates nebulization of the liquid, the ensuing aerosol particles undergo collisions with the residual liquid, leading to their disintegration, a phenomenon previously elucidated. FIG. 15b depicts the frontal view of the nebulizer to illustrate the spatial relationships among the nebulizer elements.

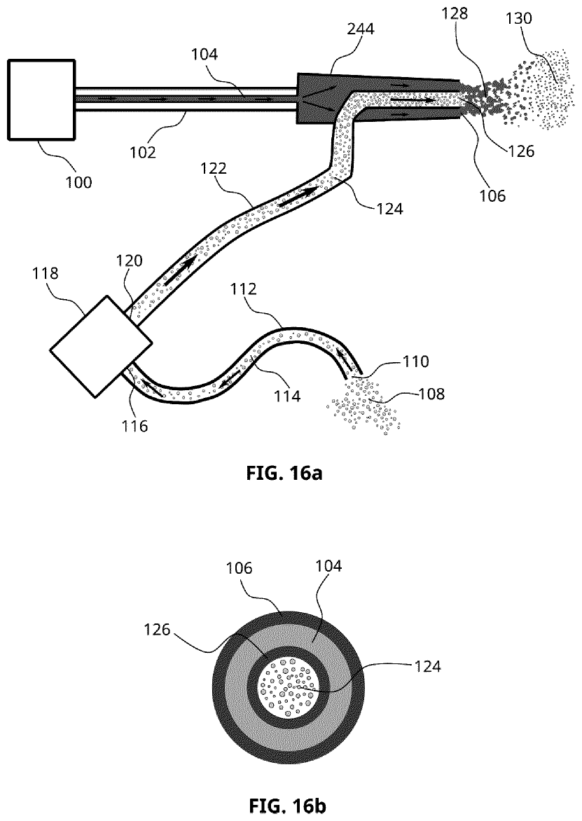

The nebulizer design discussed herein, while effective for liquid nebulization, presents a limitation in our specific application: aerosol particles predominantly exit at the periphery of the nebulizer conduit. Consequently, a considerable quantity of these particles may be diverted from the central axis prior to substantial interaction with the liquid. To address this, an alternative concentric nebulizer configuration is presented, wherein the positions of the liquid and nebulizing gas capillaries are inverted; specifically, the nebulizing gas traverses the inner capillary, while the liquid is channeled through the outer one. This design is illustrated in FIG. 16a, which echoes the structural arrangement and function of the components as delineated in FIG. 1, maintaining annotation consistency for clarity. The design also integrates an additional housing unit 244, encapsulating the terminal segments of both the liquid and nebulizing gas capillaries.

In this configuration, aerosol particles are ejected along the central axis of the concentric nebulizer, where liquid concentration is at its peak. This alignment fosters maximum collisional impact between the aerosol particles and the liquid, thereby enhancing the disintegration of the aerosol particles. Furthermore, as the aerosol particles are enveloped within the liquid volume, the majority are retained within the resultant liquid spray. This containment significantly augments the dissolution efficiency of the aerosol material. FIG. 16b offers a frontal view of the nebulizer, delineating the spatial interplay among the nebulizer components for a comprehensive understanding.

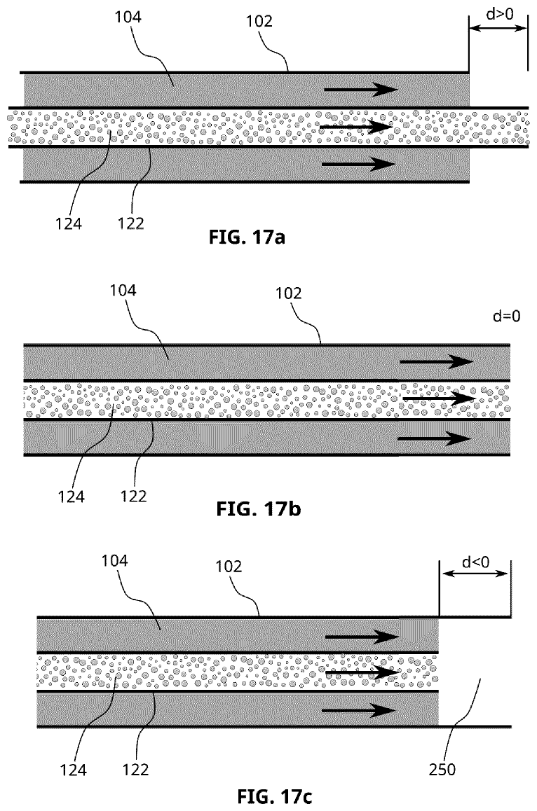

In refining concentric nebulizer designs, an important element is determining the offset (d) between the liquid and nebulizing gas capillaries. This offset can be positive, neutral, or negative, as illustrated in FIG. 17a-c, with a negative offset indicating that the outlet of the nebulizing gas conduit is recessed within the liquid capillary. The annotations for other elements in FIG. 17a-c correspond to those established in FIG. 16a. Notably, a negative offset (d<0) creates a space 250 in the liquid conduit ahead of the nebulizing gas outlet, serving as a nebulization chamber as shown in FIG. 17c. In this case, the first interaction between the nebulizing gas and the liquid happens immediately in front of the nebulizing gas conduit so the cross-section of the liquid conduit at the location of the outlet of the nebulizing gas conduit can be considered as the effective outlet of the liquid conduit. The confined nature of the space 250 enhances the nebulization disintegration effects due to prolonged gas-liquid interactions under high pressure, as previously discussed. The choice of offset influences a balance between several key factors: the overall efficiency of the nebulizer, the intensity of interaction between aerosol particles and the liquid, and, in cases where the liquid is charged, the ionization efficacy of the aerosol molecular species. Given the complex and non-linear nature of this process, a systematic approach to system optimization is advisable. This involves conducting a series of performance characterizations, followed by incremental adjustments of the parameters, in pursuit of the most favorable operational outcome, as previously discussed.

An additional enhancement often incorporated into established pneumatic ion source devices is the integration of a hot gas stream, mixed with the liquid spray. The primary objective of this modification is to expedite the evaporation of liquid droplets, thereby facilitating the rapid release of analyte molecules and/or ions. This process is instrumental in augmenting ion collection efficiency. Typically, the hot gas employed is dry nitrogen, with a flow rate generally about ten to twenty times that of the nebulizing gas, as a general guideline. Determining the optimal temperature of the hot gas involves considering the enthalpy of evaporation of the solvent used in nebulization, along with its flow rate. It is crucial to calibrate the temperature to avoid thermal degradation of the analyte while ensuring complete evaporation of the solvent. These temperature ranges are within the knowledge base of those skilled in the field. For instance, when using a predominantly water-based solvent at a flow rate of 200 μl/min, the hot gas is typically heated to approximately 250° C.

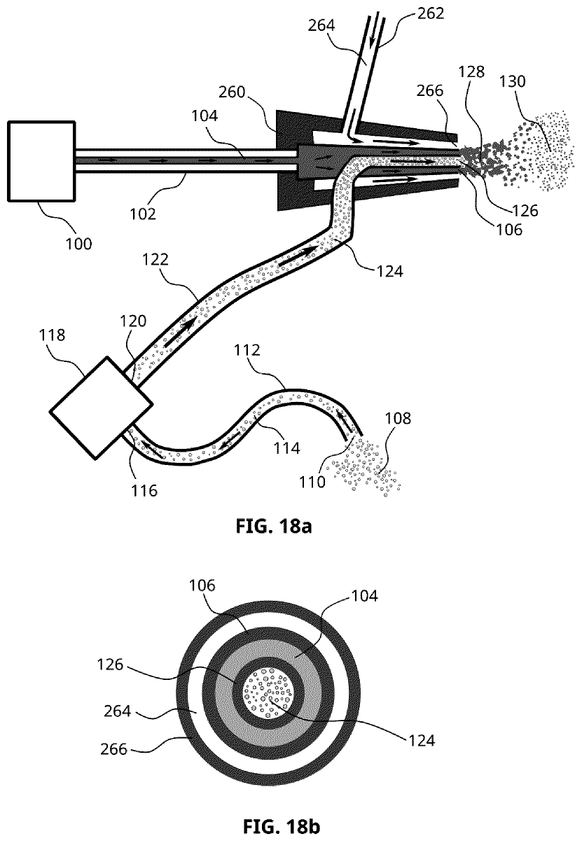

FIG. 18a illustrates an exemplary embodiment incorporating a hot gas modification, as an extension of the design depicted in FIG. 17a. In this configuration, hot gas 264 (typically dry nitrogen) is introduced via a conduit 262, which is arranged concentrically and external to the liquid conduit. The outlets of all conduits are harmoniously integrated into a housing 260. The hot gas is emitted through an outlet 266, situated near the nebulization region 128. Here, due to the Bernoulli effect, the hot gas is efficiently entrained and subsequently merges into the nebulizing gas jet. This integration facilitates a swift mixing with the liquid droplets, thereby significantly accelerating their evaporation. For an enhanced visual comprehension, FIG. 18b provides a frontal view of the nebulizer, specifically highlighting the spatial relationship and interaction among the various components within the nebulizer assembly.

Another advantage of this design is that the gas that is introduced through the concentric outlet 266 can also contribute to the nebulization of the liquid if its flow rate is high enough.

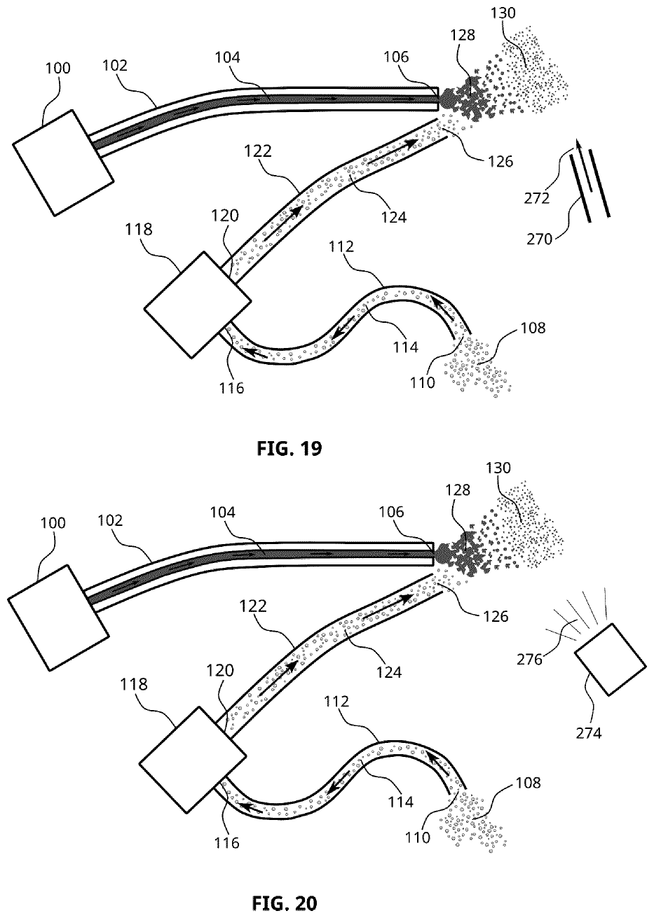

The hot gas conduit 270 can be configured to deliver the hot gas 272 with different geometries relative to the nebulization region 128 as shown in FIG. 19.

The liquid droplets can be also heated for accelerated evaporation using an infrared lamp 274 as shown in FIG. 20. If the characteristic infrared wavelength of the radiation 276 that the lamp emits is close to one of the optical absorption peaks of the liquid, illumination of the liquid droplets by that source will heat the droplets and hance contribute to their accelerated evaporation.

The nebulization process, as previously outlined, initiates with the collision of the nebulizing gas against the liquid, leading to the primary disintegration of aerosol particles. However, this mechanical impact can be just the initial step of a multifaceted process. The nebulization can also induce a range of additional phenomena that further contribute to the disintegration of aerosol particles. These phenomena, particularly effective in designs that render them thermodynamically favorable, include but are not limited to liquid fractures arising from tensile stresses, cavitations, phase explosions, and spinodal decompositions. Such events occur when the thermodynamic coordinates of a specific location within the liquid align within a certain region of the thermodynamic phase diagram for that liquid.

The inherently vigorous nature of nebulization subjects the liquid to repeated collisions, producing acoustic transients through both mechanical impact and turbulence. These transients can precipitate rapid pressure variations within the liquid volume, setting off a cascade of thermodynamic responses. For instance, cavitation, a notable response, generates secondary acoustic transients. These secondary transients, in turn, can induce further thermodynamic reactions, creating a self-perpetuating cycle. Consequently, these amplified acoustic phenomena can significantly enhance the disintegration of aerosol particles, playing a role in the efficacy and dynamics of the nebulization process.

However, it is noted that excessive violence in these secondary thermodynamic phenomena may inadvertently alter the aerosol material in an undesirable manner. For instance, the collapse of cavitation bubbles can engender extreme localized conditions, characterized by very high temperatures, pressures, and shock waves. Such intense environments are capable of breaking chemical bonds, leading to the molecular fragmentation of the aerosol material. While such pronounced cavitation bubble collapses are generally not observed in laboratory pneumatic devices, particularly in LC-MS systems equipped with robust pneumatic nebulizers, careful examination of these phenomena is recommended when devising new nebulizer designs.

In the aftermath of nebulization, a common occurrence is the broad dispersion of liquid droplets, primarily attributed to the expansion of the high-pressure nebulizing gas upon its exit from the nebulizing gas conduit. Such dispersion, however, often leads to the loss of analyte and a consequent reduction in the strength of the analytical signal, which is generally undesirable in analytical applications.

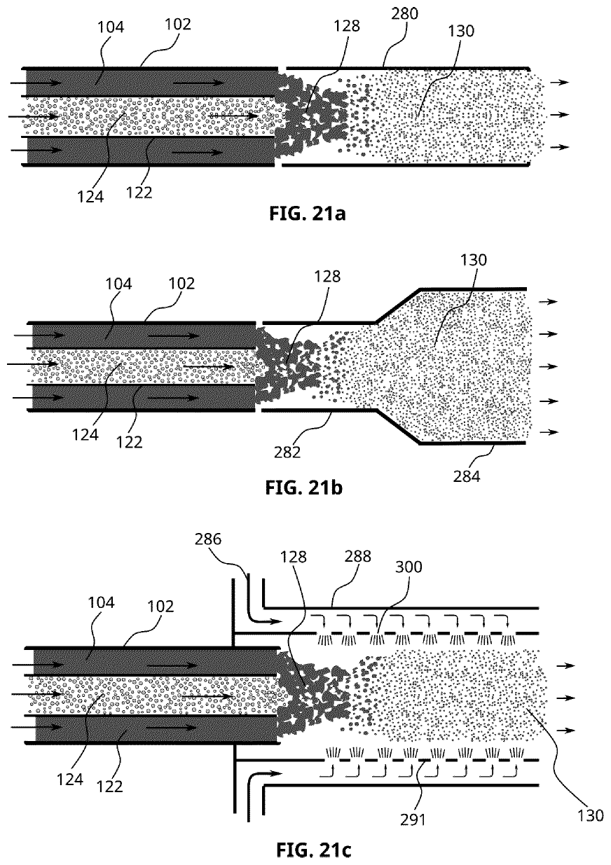

To mitigate this issue, the introduction of an additional component, termed a ‘spray conduit’, 280 as depicted in FIG. 21a, can be beneficial. This design of the spray conduit parallels the elements 102, 104, 122, 124, 128, 130, consistent with those in the previously described embodiments. The strategic placement of the inlet of the spray conduit encompasses most of the nebulization volume 128, effectively directing the expanded nebulizing gas—and consequently, the aerosol material—predominantly towards the outlet of spray conduit. This orientation ensures that the location of the aerosol material remains confined within the spray conduit and its outlet.

The geometric design of the spray conduit, including its outlet, can be specifically tailored to meet the requirements for efficient aerosol material delivery. An example is illustrated in FIG. 21b, where a spray conduit embodiment 282 features an outlet 284 with an expanded cross-sectional area. This particular design is advantageous for achieving a more laminar flow of the aerosol-laden gas stream, facilitating a more controlled and stable integration of aerosol particle fragments and aerosol molecular species into an analytical instrument.

A notable consideration in this configuration is the potential for liquid droplet condensation along the inner walls of the spray conduit. To address this, the incorporation of hot gas into the spray conduit is recommended to expedite the evaporation of these droplets. This can be achieved longitudinally via a concentric secondary gas conduit, as described in relation to FIG. 18a. An alternative approach is depicted in FIG. 21c, showcasing a design where a hot gas conduit 288 is concentrically aligned with the spray conduit 291. The spray conduit, characterized by a perforated wall and encapsulated within the hot gas conduit, allows the hot gas 286 to permeate the interior of the spray conduit through the perforations 300. This not only aids in droplet evaporation but also effectively prevents droplet accumulation on the inner walls of the conduit, as the flow of hot gas through the perforations actively blows off the droplets from the inner wall of the spray conduit.

Further enhancing the spray conduit design involves the implementation of heating of the spray conduit. This modification, familiar to those skilled in the art, involves maintaining the spray conduit at an elevated temperature. The primary advantage of this approach lies in the increased evaporation rate of the spray droplets as they traverse the conduit. Consequently, this accelerated evaporation process is conducive to a higher rate of ion desorption.

Heated capillaries, akin to the proposed heating configuration, are routinely employed at the inlets of conventional mass spectrometers equipped with atmospheric ion sources. Their primary function in such contexts is to swiftly eliminate any residual droplets incoming from external sources, such as those generated by an electrospray. The design and operational principles of such heated capillaries are well-established and widely recognized by experts in the field.

As detailed in the Summary, aerosol disintegration can yield varied results. A portion of aerosol particles may break into smaller fragments, another portion may decompose into molecular aerosol species, while some particles may remain unchanged. For a specific aerosol type, the specific ratio of these fractions in the resulting aerosol material depends primarily on the nebulization system's parameters, including the type of liquid, liquid flow rate, nebulizing gas flow rate, pressure in the nebulizing gas conduit, and the nebulizer's geometry. Adjusting these parameters allows for the optimization of the aerosol disintegration outcome based on the characteristics of the aerosol particles.

In the context of the present disclosure when the aerosol disintegration process produces at least some molecular species and where the objective is to analyze these aerosol molecular species using a mass spectrometer, the ionization of these species is an important step. A wide range of ionization methods are applicable in such scenarios. Practically, the ionization of molecules typically occurs either in a vacuum or under atmospheric pressure.

When operating in a vacuum, common ionization techniques include Electron Ionization (EI) and Direct Photoionization (PI). Photoionization can be achieved through single-photon UV absorption, or by employing more advanced multi-photon techniques like Resonance Enhanced Multi-Photon Ionization (REMPI). A significant advantage of direct photoionization methods lies in their deterministic nature and the potential for selectivity, achieved by tuning the optical properties of the ionizing light. This selectivity is invaluable for quantitative measurements, which, while crucial, often present significant challenges. However, it is noted that direct photoionization methods are typically effective only for molecules with relatively small molecular masses. The practical upper limit for these methods usually lies around 3000 Daltons (Da).

Atmospheric pressure ionization techniques are much more ubiquitous in bio-medical research because of their simplicity, compatibility with complex biological samples, ability to soft ionize large biomolecules, high sensitivity, and minimal sample preparation. The most popular atmospheric pressure ionization methods are electrospray ionization (ESI), atmospheric pressure chemical ionization (APCI), and atmospheric pressure photoionization (APPI).

The conventional ESI mechanism begins with a solution of the analyte being injected through a fine needle to create a mist. This needle is held at a high voltage, creating an electric field that imparts a charge to the droplets in the mist. As these charged droplets move through the electric field, they are subjected to both evaporation and Coulombic fission which causes the droplets to become progressively smaller. This process continues until the droplets reach a point where they can no longer hold their solvent and the analyte molecules, now ionized, are ejected into the gas phase.

Electrospray ionization (ESI) fundamentally requires only a high voltage supply and a capillary for effective operation. Two conditions are essential for ESI: First, the liquid within the system must contact a conductor, which is charged to a high electric potential, either positive or negative. Second, a significant electric field gradient must exist at some point within the liquid conduit, facilitating charge separation and electrochemistry. The site of this intense electric field gradient need not be near the conductor since the conductor can be electrically shielded; Typically, in conventional ESI systems, most of the electrochemistry and liquid charging occurs at the tip of the capillary, where the electric field is strongest. While early ESI devices employed metal capillaries to establish this gradient, later innovations have successfully utilized non-conductive capillaries. This advancement is possible because the solvents themselves act as conductors, naturally creating an electric field at the capillary tip.

When liquid flow rates are high, the electric field alone is not sufficient to break the liquid into a fine spray. In those cases, pneumatic nebulizers are used in addition to electric fields to create the electrospray. This general method provides a simple and effective approach for ionizing aerosol molecular species in our case since it is a natural extension of our embodiments.

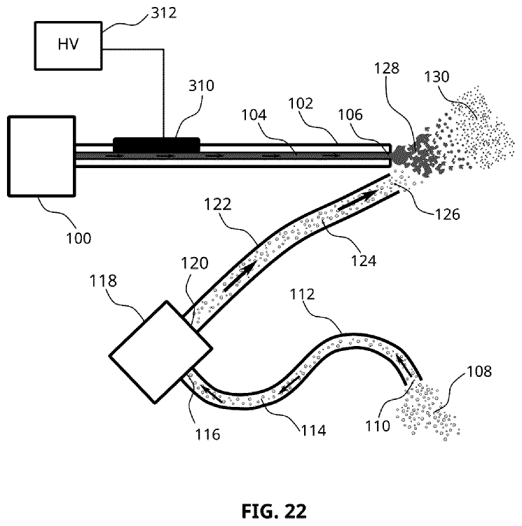

FIG. 22 demonstrates the implementation of ESI ionization in our designs, consistent with the elements and reference numbers detailed in FIG. 1. Key additions include an electrical conductor 310, designed to maintain electrical contact with the liquid 104 within the liquid conduit 102, and a high voltage supply 312, connected to the conductor 310. Specific electrical configurations, such as grounding, are omitted as they are standard knowledge for those skilled in the art. In this setup, the electrically charged liquid exits the conduit 102, where it is nebulized by the nebulizing gas that carries the aerosol particles. The aerosol molecular species that get generated as a consequence of the nebulization get further ionized as molecules in the conventional electrospray devices.

Optimizing the pneumatic ESI operation in our device presents a more intricate challenge than in conventional ESI systems, due to the dual objectives of enhancing both aerosol disintegration and ESI efficiency, which are interdependent on the nebulization process. For example, in the case of concentric nebulizers a factor in this optimization is the offset between the gas and liquid capillaries, which is illustrated in FIG. 17a-c. This offset not only affects the electric field intensity experienced by the liquid, impacting the ESI process, but also significantly influences the aerosol disintegration efficiency. Other key design considerations include the material selection for the capillaries and their respective internal and external diameters. An effective approach to optimization might involve starting with a proven baseline design, such as that shown in FIG. 17a, where both capillaries are constructed from stainless steel. Subsequent adjustments to various parameters can be made while continuously evaluating the overall analytical performance of the device. Supplementing this empirical approach with numerical simulations for fluid dynamics and electric fields can offer valuable insights and aid in fine-tuning the design of the device.

On the other hand, the fact that a clean liquid is used for nebulization in our approach provides some advantages compared to conventional ESI. Traditional ESI systems employ a liquid containing an analyte, leading to potential deposit buildup on the capillaries over time due to electrochemistry that occurs between the analyte and the capillary. This can affect the consistency and continuity of the ionization signal. Our method mitigates such challenges by using a clean nebulizing liquid, enhancing the device's operational efficiency and reliability.

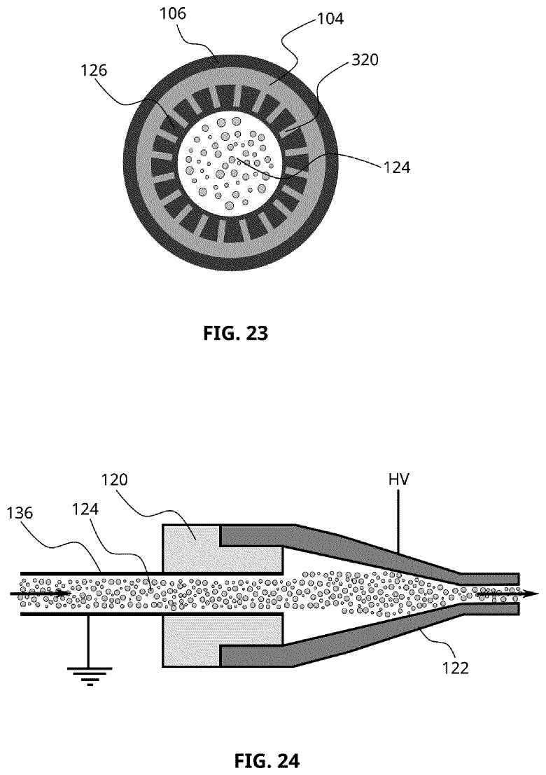

FIG. 23 illustrates an enhancement to the liquid conduit outlet of the concentric nebulizer, as previously detailed in FIG. 16a-b, aimed at improving ionization efficiency. In this design, the inner capillary, which conducts the nebulizing gas, is constructed from stainless steel, and is placed at a high electric potential. A key feature in this embodiment involves the incorporation of micromachined channels 320 on the outer surface of the capillary at its outlet. These channels serve to expand the surface area available for electrochemistry, occurring between the liquid traversing the inner capillary and the capillary itself. This expansion enhances the efficiency of liquid charging. Furthermore, a higher concentration of charges in the liquid enhances the ionization process, leading to more effective ionization of the aerosol molecular species. Micromachining of spatial features on this scale can be readily accomplished with laser micromachining tools. The presence of such channels without compromising the signal stability is possible because of clean liquids in our case. The geometry of the micromachining feature presented in FIG. 23 is non-limiting. Other micromachining features with various geometries are possible. The key concept is to increase the area of the contact surface between the liquid and the conductor where most of the electrochemistry takes place in order to charge the liquid more efficiently.

An additional optimization strategy based on leveraging the use of clean liquids to enhance the signal-to-noise ratio is feasible when aerosol particles are generated in a pulsed manner, such as with lasers. This method entails pulsing the electrical potential applied to the conductor in contact with the liquid. When aerosol production duty cycles are significant, maintaining a constant electrical voltage at the capillary is unnecessary in the absence of aerosol particles at the nebulizer. Unneeded charging of the liquid during these intervals can introduce background noise. Synchronizing the pulsing of the high electric potential with the arrival of new aerosol particles ensures that the voltage is active only when needed, thereby reducing noise and enhancing signal clarity.

In addressing the challenge of coupling aerosol particles from the gas coupler outlet to the high-voltage nebulizer gas conduit, a key consideration is the interface between these two components, as defined in relation to FIG. 2. Our previous embodiments recommend grounding the aerosol transport system to prevent static electricity issues. The question arises: how can we effectively interface the grounded gas coupler with the high-voltage nebulizer gas conduit? FIG. 24 introduces a solution with the interface 120 between the grounded outlet of the gas coupler 136 and the high-voltage nebulizer gas conduit 122. Here, the gas coupler outlet ends with a grounded conductor, passing through a ceramic interface 120. The inlet of the nebulizer gas conduit, being a conductor at high voltage, surrounds this interface. This design ensures a sufficient gap between the gas coupler outlet and the inner wall of the nebulizer conduit, larger than the dielectric breakdown distance, typically a few millimeters.

Within this setup, aerosol particles 124 are electrically shielded in the grounded outlet 136 until they are rapidly injected into the nebulizer gas conduit. With carefully chosen interface parameters, the kinetic energy of the gas jet passing through the interface 120 can surpass any boundary electric fields, thereby preserving the presence of even previously charged or polarized aerosol particles while maintaining the whole device safe to operate.

Integrating APCI and APPI ionization methods with our embodiments is more straightforward compared to ESI. This is because, in APCI and APPI, the ionization process is decoupled from nebulization. Once aerosol molecular species are generated as per our methods, APCI and APPI can be employed using techniques familiar to those skilled in the field. For simplicity, these well-known technical details are skipped in the descriptions below.

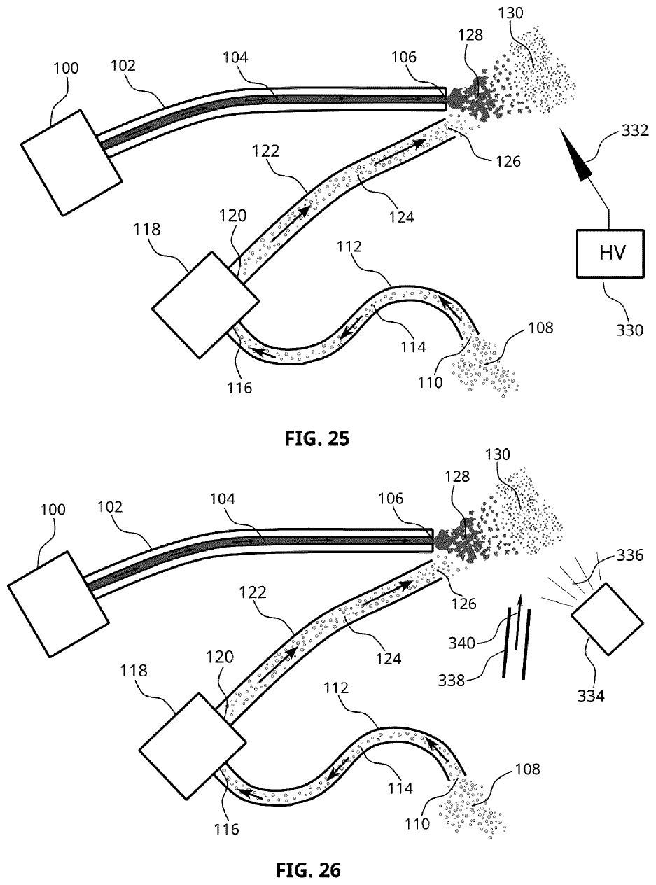

FIG. 25 presents a streamlined configuration for integrating APCI into our designs. The component functions and references align with those outlined in FIG. 1. The key addition is the corona discharge needle 332, connected to a high voltage supply 330 and optimally located near the emergence point of the aerosol molecular species. As these species traverse the corona region, they undergo ionization through interaction with ions produced by the corona discharge.

FIG. 26 presents a simplified embodiment for APPI integration. In this embodiment, a donor gas is combined with the aerosol molecular species. This mixture is then exposed to ultraviolet (UV) light 336 from a UV lamp 334, which ionizes the donor gas. The ionized donor gas subsequently transfers its charge to the aerosol molecular species, resulting in their ionization.

For APPI, a design choice must be made about the method of introducing the donor gas into the volume where the aerosol molecular species are located. FIG. 26 illustrates the donor gas 340 being supplied via a separate source 338. However, our designs offer a more streamlined approach. One option involves incorporating a liquid component, like toluene, into the liquid 104, which, upon nebulization and evaporation, gets ionized by the UV lamp. An alternative is introducing the donor gas through a secondary gas source 180, as shown in FIG. 7. In both scenarios, the nebulization effectively blends the donor substance with the aerosol material, enhancing APPI efficiency.

In the implementation of both APCI and APPI, ensuring the majority of the liquid evaporates prior to ionization can be beneficial. Methods for enhancing liquid evaporation have been detailed earlier in this disclosure. For clarity, these methods are not depicted in FIG. 25 and FIG. 26.

The present disclosure offers the added benefit of facilitating straightforward signal calibration across various ionization methods. During calibration, the nebulizing gas remains uncontaminated, while calibration substances are dissolved in the liquid 104. Given that the aerosol disintegration is a reliable mechanical process, independent of ionization and ion collection efficiencies, the signal intensities produced by the calibration substances are indicative of those expected from aerosol analysis. Additionally, for real-time signal calibration, the calibration substance can be introduced alongside the aerosols into the nebulizing volume.

Ion collection in atmospheric conditions poses a challenge for mass spectrometry, as gas currents can significantly influence ion trajectories, making them as impactful as electric fields and harder to control. This has historically resulted in lower ion collection efficiencies in conventional LC-MS systems with pneumatic nebulizers, averaging about 1-5%. However, recent advancements have improved these efficiencies. Ion optics, traditionally used in low-pressure conditions within mass spectrometers, employ various electrode configurations, like ring electrodes, to guide ions through their central apertures. These electrodes are subjected to static or transient DC and RF voltages. A notable configuration is the ion funnel, consisting of stacked ring electrodes with progressively decreasing diameters. The design of ion funnels is well-established in the field.

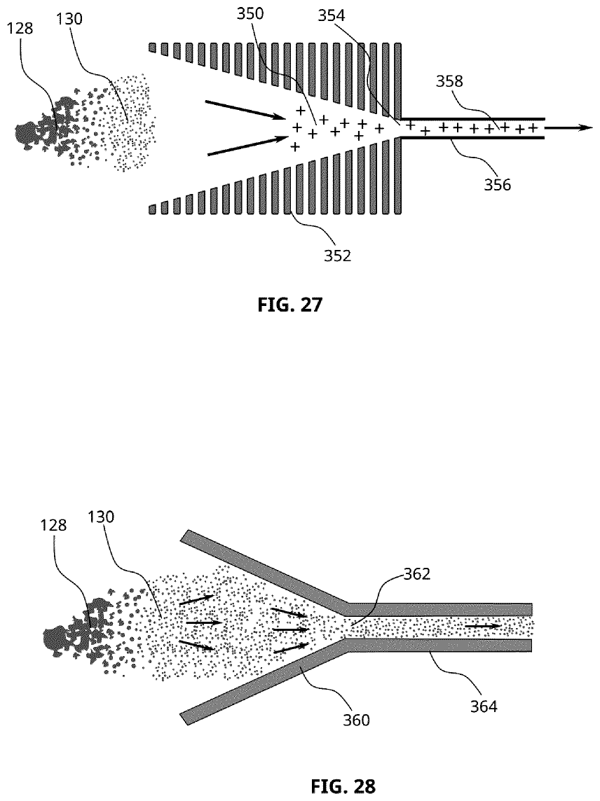

Recent developments have shown ion funnels to be effective under atmospheric conditions as well, significantly enhancing ion collection efficiency. Such designs are applicable in our embodiments. FIG. 27 illustrates the integration of an ion funnel in our system. The aerosol ions 350, formed as a consequence of nebulization within the volume 128 and propelled within the mixture 130 by the gas stream, are intercepted by the ion funnel 352. This funnel directs the ions through its exit aperture 354, after which an ion guiding conduit 356 channels the collected ions 358 into the mass spectrometer.

In certain scenarios, analyzing neutral aerosol particle fragments and aerosol molecular species produced during nebulization can be valuable. For these instances, as depicted in FIG. 28, a simple gas funnel can be employed for collection. The aerosol particle fragments and aerosol molecular species generated as a consequence of the nebulization within the nebulizing region 128 and carried by the mixture 130 in the gas stream are gathered by a gas funnel 360. This funnel, aided by its collection cone and the gas flow, directs the aerosol particle fragments and aerosol molecular species toward its aperture 362 and subsequently through the funnel conduit 364 for analysis.

In some applications, optical methods of examination also offer significant advantages. Optical excitations, typically carried out using lasers or focused light sources, benefit from having conduits that restrict the movement of ions or neutral aerosol particle fragments and aerosol molecular species within specific dimensions. We have previously described such conduits in this disclosure, following the ion and gas funnels, to facilitate these analyses.

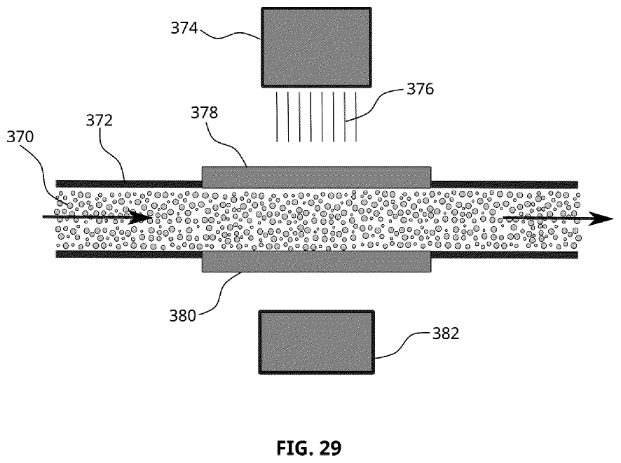

Turning to FIG. 29, we present an exemplary embodiment showcasing an optical excitation setup for aerosol particles 370 passing through a conduit 372. This setup includes an optically transparent window 378 integrated into the wall of the conduit, allowing for the passage of optical radiation 376 from an external source 374. The radiation penetrates through the window 378, interacting with aerosol particles inside the conduit. For detecting optical signals, another transparent window 380 can be incorporated opposite the first, enabling external detection by an apparatus 382. This design allows for precise optical excitation and efficient signal capture, enhancing the potential for detailed optical analysis.

The methodologies of the present disclosure are particularly applicable in analyzing aerosols generated from laser plumes. While the present disclosure does not delve into the specifics of laser ablation or its varied applications, as they are extensively covered in existing literature, we will highlight a selection of generic applications where the techniques disclosed in the present disclosure can be effectively utilized.

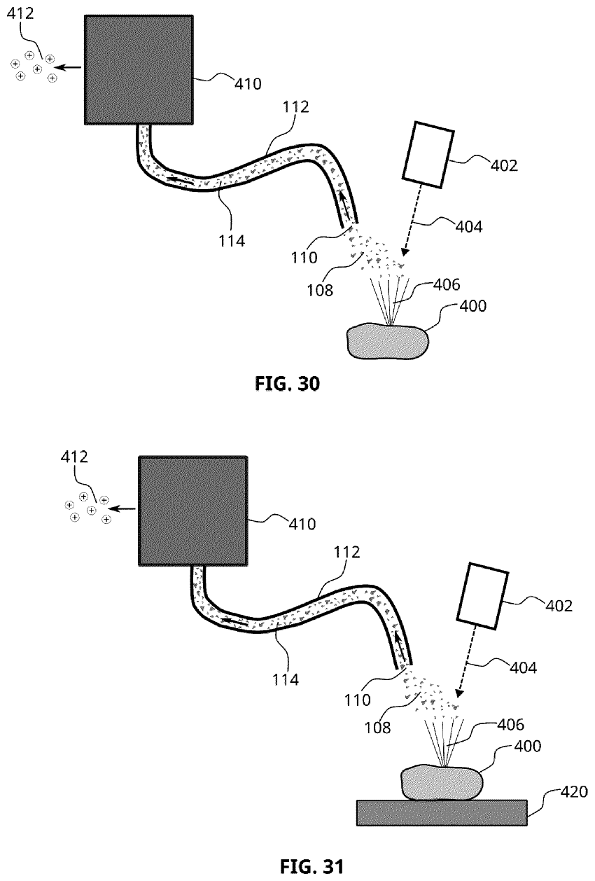

FIG. 30 exemplifies a typical setup, presented in a non-restrictive manner. In this scenario, a condensed phase sample 400 undergoes ablation by a laser 402, with the laser beam directed via delivery optics 404. This ablation process generates a laser plume 406, from which aerosol particles 108 are partially or wholly captured by an aspirator inlet 110. These particles 114 are then transported through an aspirator conduit 112 to an analysis system 410. This system, representing one of the previously described embodiments in the present disclosure for aerosol analysis, processes the particles to produce ions 412, which are subsequently analyzed by a mass spectrometer.

The ablation laser features a peak emission wavelength ranging from 100 nm to 12 μm, optimally tuned for strong absorption by the sample, thereby localizing energy deposition effectively. The laser typically operates in a pulsed mode, with pulse durations spanning 10 fs to 1 ms. Shorter pulses are generally preferred to minimize residual damage in the sample. This technique is applicable to a variety of condensed matter samples, including both in vivo and ex vivo biological tissues.

FIG. 31 depicts a variation of the embodiment shown in FIG. 30. In this setup for sample imaging, the sample 400 is mounted on a motorized translation stage 420. This allows for sampling at multiple spatial points on the sample, correlating each point with its specific location. The resulting laser plumes from these points are then analyzed using system 410. This approach enables the determination of the spatial distribution of specific molecular species within the sample.

The sample imaging analysis depicted in FIG. 31 can be multiplexed with other imaging methods. Notably, combining it with optical imaging methods like vibrational spectroscopy, such as Stimulated Raman Scattering (SRS) imaging or Coherent Anti-Stokes Raman Scattering (CARS) imaging, offers significant advantages. These methods provide detailed molecular insights by exploiting the Raman scattering process, where photons interact with molecular vibrations. SRS and CARS imaging can guide the laser sampling spot selection with their high-resolution molecular images, enabling precise targeting of specific sample areas. Additionally, they furnish complementary information about the chemical and structural makeup of the sample. This integrated approach enriches the analysis obtained from mass spectrometry of aerosol particles, leading to a more comprehensive understanding of the sample. The benefits of vibrational spectroscopy and instrumentation details are well documented in academic literature, emphasizing its role in enhancing molecular specificity and spatial resolution in sample analysis.

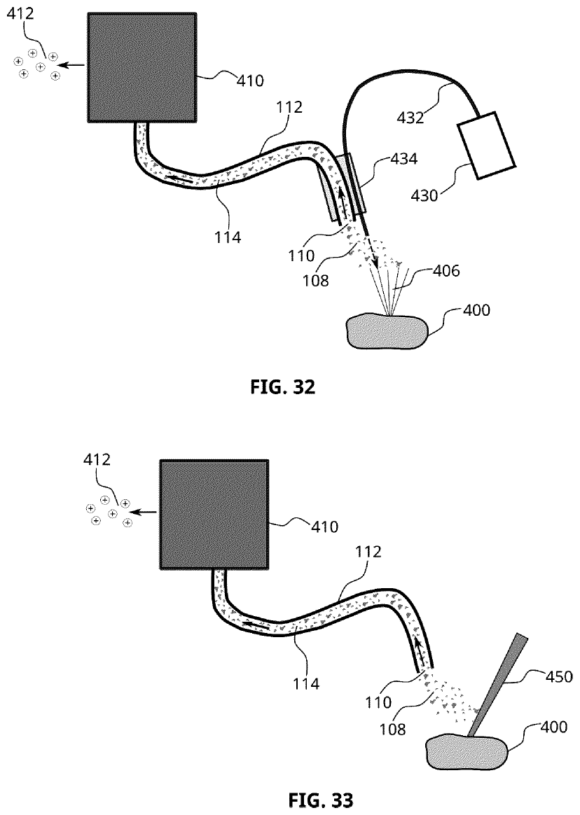

FIG. 32 presents an alternate version of the generic embodiment shown in FIG. 30. In this design, the laser light is directed to the sample 400 via an optical fiber 432. The output of the fiber and the aspirator inlet 110 are combined within a single housing unit 434. This housing is designed to be ergonomic, allowing for handheld use.

In addition to lasers, alternative methods can generate aerosols from biological tissues. FIG. 33 depicts a scenario where an electrosurgical tool 450 is employed to aerosolize a sample 400. This aerosol is then analyzed using system 410, as previously outlined in the context of FIG. 30.

Once ions are created from aerosol particles by non-limiting embodiments described in the present disclosure, these ions can be analyzed by a mass spectrometer. Mass spectrometer designs as well as types and methods of mass spectrometry measurements are well known to a person skilled in the art. Standard mass spectrometers that can be used include time-of-flight (TOF) mass spectrometers, quadrupole mass spectrometers, ion trap mass spectrometers, Orbitrap mass spectrometers, triple quadrupole mass spectrometers, Fourier transform ion cyclotron resonance (FT-ICR) mass spectrometers, and various hybrid systems.

The disclosed embodiments facilitate versatile spatial configurations in which aerosol molecular species are initially generated at a first location, subsequently transported to a second location for ionization, and ultimately the resulting ions are collected and analyzed using a mass spectrometer at a third location. We will just describe a generic configuration for systems that include the present disclosure and a mass spectrometer.

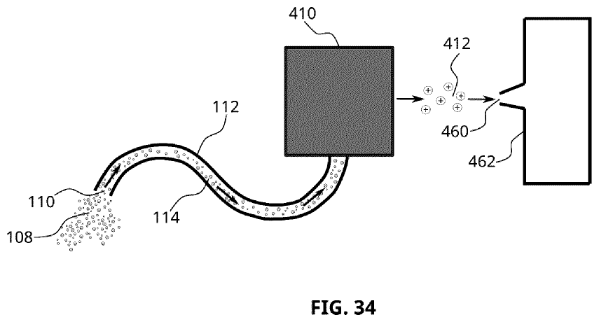

In FIG. 34 a non-limiting configuration is illustrated with a system 410 encompassing one or more embodiments of the present disclosure, where the system 410 produces ions 412 from sample aerosol particles 108 that are collected using an aspirator conduit 114 with an inlet 110, where the ions are generated in vicinity of the orifice 460 of a mass spectrometer 462, and where the ions are collected and analyzed by a mass spectrometer. The system 410 may contain an ion collection and ion transportation systems that efficiently transfer the ions into the orifice 460.

A potential enhancement over conventional mass spectrometry instruments involves the incorporation of an ion drift tube, as detailed in the Summary section of the present disclosure. This tube facilitates ion separation based on both gas mobility and mass-to-charge ratios. This enhancement mirrors the analytical capabilities of LC-MS systems, proving particularly beneficial in analyzing complex biological samples.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

### REFERENCES

[1] S. Amini-Nik et al., “Ultrafast Mid-IR Laser Scalpel: Protein Signals of the Fundamental Limits to Minimally Invasive Surgery,” PLOS ONE, vol. 5, no. 9, p. e13053, September 2010
[2] K. Franjic, M. L. Cowan, D. Kraemer, and R. J. D. Miller, “Laser selective cutting of biological tissues by impulsive heat deposition through ultrafast vibrational excitations,” Opt. Express, vol. 17, no. 25, pp. 22937-22959, December 2009
[3] M. Wurlitzer et al., “Mass Spectrometric Lipid Profiles of Picosecond Infrared Laser-Generated Tissue Aerosols Discriminate Different Brain Tissues,” Lasers in Surgery and Medicine, vol. 52, no. 3, pp. 228-234, 2020.
[4] F. Cao, F. Donnarumma, and K. K. Murray, “Particle size measurement from infrared laser ablation of tissue,” Analyst, vol. 141, no. 1, pp. 183-190, 2016.
[5] B. Fatou et al., “In vivo real-time mass spectrometry for guided surgery application,” Scientific reports, vol. 6, p. 25919, 2016.
[6] M. Kwiatkowski et al., “Ultrafast extraction of proteins from tissues using desorption by impulsive vibrational excitation,” Angewandte Chemie International Edition, vol. 54, no. 1, pp. 285-288, 2015.
[7] E. Nikolaev and J. Franzen, “Mass spectrometry with laser ablation,” U.S. Pat. No. 7,910,881B2, Mar. 22, 2011
[8] U. Pöschl, “Aerosol particle analysis: Challenges and progress,” Analytical and bioanalytical chemistry, vol. 375, pp. 30-2, February 2003
[9] J. Ho, “Future of biological aerosol detection,” Analytica Chimica Acta, vol. 457, no. 1, pp. 125-148, 2002.
[10] U. Pöschl, “Atmospheric Aerosols: Composition, Transformation, Climate and Health Effects,” Angewandte Chemie International Edition, vol. 44, no. 46, pp. 7520-7540, 2005
[11] E. C. Ellis and T. Novakov, “Application of thermal analysis to the characterization of organic aerosol particles,” in Studies in Environmental Science, vol. 20, Elsevier, 1982, pp. 227-238
[12] R. B. Dixon, J. S. Sampson, A. M. Hawkridge, and D. C. Muddiman, “Ambient aerodynamic ionization source for remote analyte sampling and mass spectrometric analysis,” Analytical chemistry, vol. 80, no. 13, pp. 5266-5271, 2008.
[13] Y. H. Rezenom, J. Dong, and K. K. Murray, “Infrared laser-assisted desorption electrospray ionization mass spectrometry,” Analyst, vol. 133, no. 2, pp. 226-232, 2008.
[14] A. Vertes and P. Nemes, “U.S. Pat. No. 8,809,774-Laser ablation electrospray ionization (LAESI) for atmospheric pressure, in vivo, and imaging mass spectrometry,” U.S. Pat. No. 8,809,774B2, Aug. 19, 2014
[15] E. A. Jones, D. Simon, T. Karancsi, J. Balog, S. D. Pringle, and Z. Takats, “Matrix assisted rapid evaporation ionisation mass spectrometry,” Analytical chemistry, 2019.
[16] Z. Takats, “Collision surface for improved ionisation,” U.S. Pat. No. 11,342,170B2, May 24, 2022
[17] J. Balog, T. Karancsi, S. D. Pringle, Z. Takats, J. Kinross, and J. K. Nicholson, “In vivo endoscopic tissue identification tool,” U.S. Pat. No. 11,139,156B2, Oct. 5, 2021
[18] R. Javanshad and A. R. Venter, “Ambient ionization mass spectrometry: real-time, proximal sample processing and ionization,” Analytical Methods, vol. 9, no. 34, pp. 4896-4907, 2017.
[19] A. Amirav, T. Shahar, and S. Dagan, “Method and apparatus for sample introduction into a mass spectrometer for improving a sample analysis,” U.S. Pat. No. 5,742,050A, Apr. 21, 1998
[20] B. Fatou et al., “Remote atmospheric pressure infrared matrix-assisted laser desorption-ionization mass spectrometry (remote IR-MALDI MS) of proteins,” Molecular & Cellular Proteomics, vol. 17, no. 8, pp. 1637-1649, 2018.
[21] T. R. Covey, B. A. Thomson, and B. B. Schneider, “Atmospheric pressure ion sources,” Mass spectrometry reviews, vol. 28, no. 6, pp. 870-897, 2009.
[22] J. A. Burgener and Y. Makonnen, “Nebulization systems,” in Sample introduction systems in ICPMS and ICPOES, Elsevier, 2020, pp. 57-142.
[23] A. Kruve, I. Leito, K. Herodes, A. Laaniste, and R. Lohmus, “Enhanced nebulization efficiency of electrospray mass spectrometry: improved sensitivity and detection limit,” Journal of the American Society for Mass Spectrometry, vol. 23, no. 12, pp. 2051-2054, 2012.
[24] C. L. Feider, A. Krieger, R. J. DeHoog, and L. S. Eberlin, “Ambient ionization mass spectrometry: Recent developments and applications,” Analytical chemistry, vol. 91, no. 7, pp. 4266-4290, 2019.
[25] F. Gunzer, S. Krüger, and J. Grotemeyer, “Photoionization and photofragmentation in mass spectrometry with visible and UV lasers,” Mass Spectrometry Reviews, vol. 38, no. 2, pp. 202-217, 2019.
[26] L. Meier, C. Berchtold, S. Schmid, and R. Zenobi, “Extractive Electrospray Ionization Mass Spectrometry—Enhanced Sensitivity Using an Ion Funnel,” Anal. Chem., vol. 84, no. 4, pp. 2076-280 February 2012
[27] E. Ahmed, D. Xiao, K. M. M. Kabir, J. Fletcher, and W. A. Donald, “Ambient Pressure Ion Funnel: Concepts, Simulations, and Analytical Performance,” Anal. Chem., vol. 92, no. 24, pp. 15811-15817, December 2020
[28] T. C. Hollon et al., “Near real-time intraoperative brain tumor diagnosis using stimulated Raman histology and deep neural networks,” Nature medicine, vol. 26, no. 1, pp. 52-58, 2020.
[29] C. Seger and L. Salzmann, “After another decade: LC-MS/MS became routine in clinical diagnostics,” Clinical Biochemistry, vol. 82, pp. 2-11, August 2020
[30] S. C. Henderson, S. J. Valentine, A. E. Counterman, and D. E. Clemmer, “ESI/ion trap/ion mobility/time-of-flight mass spectrometry for rapid and sensitive analysis of biomolecular mixtures,” Analytical chemistry, vol. 71, no. 2, pp. 291-301, 1999.

1. A system for performing at least partial disintegration of sample aerosol particles, the system comprising:
a liquid source unit comprising a liquid conduit, the liquid conduit comprising a liquid conduit outlet, the liquid source unit being configured to dispense a liquid through the liquid conduit outlet;
a pneumatic nebulizer comprising a high-pressure nebulizing gas mechanism in gas-flow communication with a nebulizing gas conduit, the nebulizing gas conduit comprising a nebulizing gas conduit inlet and a nebulizing gas conduit outlet, wherein the nebulizing gas conduit outlet is positioned adjacent to the liquid conduit outlet to facilitate pneumatic nebulization of the liquid exiting the liquid conduit outlet by contact with a high-pressure nebulizing gas exiting the nebulizing gas conduit;
an aspirator device comprising an aspirator conduit, the aspirator conduit comprising an aspirator conduit inlet and an aspirator conduit outlet, the aspirator device being configured to generate suction suitable for drawing the sample aerosol particles into the aspirator conduit through the aspirator conduit inlet; and
a gas coupler providing gas coupling between the aspirator conduit outlet and the nebulizing gas conduit inlet, thereby facilitating the introduction of the aerosol particles into the high-pressure nebulizing gas stream, such that the nebulization process promotes collisions between the aerosol particles and the liquid, contributing to the disintegration, at least in part, of the aerosol particles.

2. The system of claim 1, wherein the liquid comprises a solvent configured to dissolve a least a portion of the aerosol material either during the nebulization process, within the liquid droplets resulting from the nebulization process, or during both stages.

3. The system according to claim 1, wherein the gas coupler includes at least one gas compressor.

4. The system according to claim 1, wherein the gas coupler includes at least one Venturi pump.

5. The system according to claim 1, wherein the gas coupler includes at least one device selected from the group consisting of: scroll compressor, diaphragm compressor, screw compressor, axial compressor, centrifugal compressor, rotary vane compressor, scroll pump, diaphragm pump, centrifugal pump, and rotary vane pump.

6. The system according to claim 1, further comprising an additional gas source of an additional gas in fluid communication with the gas coupler, such that the additional gas is introduced into the nebulizing gas conduit and mixed with the aerosol particles.

7. The system of claim 6, wherein the gas coupler comprises one of a gas compressor and a gas pump as a component, and the fluid communication between the additional gas source and the gas coupler is configured to occur before an inlet of the gas compressor or an inlet of the gas pump, with the additional gas source comprising a valve to control the flow rate of the additional gas entering the gas coupler, thereby enabling regulation of the suction pressure at the inlet of the aspirator.

8. The system of claim 6, wherein the gas coupler includes a component selected from the group consisting of a gas compressor and a gas pump, and wherein the fluid communication between the additional gas source and the gas coupler is established after an outlet of the gas compressor or an outlet of the gas pump, with the introduction of the additional gas into the nebulizing gas conduit serving to increase the overall gas flow rate within said conduit.

9. The system of claim 8, wherein the system is configured such that the additional gas introduced into the nebulizing gas conduit is configured to advantageously enhance the disintegration of aerosol particles during the nebulization process and/or to facilitate the advantageous ionization of aerosol molecular species following the nebulization.

10. The system according to claim 1, further comprising a filter, the filter being incorporated within the gas coupler and configured to limit the size of aerosol particles that reach the nebulizing gas conduit outlet.

11. The system of claim 10, wherein the filter integrated within the gas coupler is a cyclone filter.

12. The system according to claim 1, further comprising a check valve, the check valve being integrated within the gas coupler and configured to be open only when the gas within the gas coupler flows in the direction towards the nebulizing gas conduit outlet.

13. The system of claim 12, wherein the check valve integrated within the gas coupler is a Tesla valve.

14. The system according to claim 1, further comprising a hard obstacle positioned within the gas coupler, the hard obstacle being configured to induce turbulence in the mixture of nebulizing gas and aerosol particles as they pass around the obstacle, the turbulence contributing to at least partial disintegration of the aerosol particles within the mixture.

15. The system according to claim 1, further comprising a hard obstacle situated within the gas coupler and positioned in the path of the aerosol particles traversing through the gas coupler, whereupon collision of the aerosol particles with this obstacle contributes to at least partial disintegration of the aerosol particles.

16. The system according to claim 1, wherein the liquid source unit comprises a liquid reservoir and a liquid pump, with the liquid pump being configured to transfer liquid from the liquid reservoir into the liquid conduit.

17. The system of claim 16, wherein the liquid pump within the liquid source unit is a syringe pump.

18. The system according to claim 1, wherein the liquid conduit outlet and the nebulizing gas conduit outlet are integrated within a nebulizer device of a type selected from the group consisting of: cross flow nebulizer, V-Groove nebulizer, thin film nebulizer, parallel path nebulizer, enhanced parallel path nebulizer, Hildebrand grid nebulizer, and flow blurring nebulizer.

19. The system according to claim 1, wherein the liquid conduit and the nebulizing gas conduit are arranged concentrically relative to each other in the vicinity of the liquid conduit outlet or the nebulizing gas conduit outlet.

20. The system of claim 19, wherein the liquid conduit resides at least partially within the nebulizing gas conduit.

21. The system of claim 19, wherein the nebulizing gas conduit resides at least partially within the liquid conduit.

22. The system according to claim 1, further comprising a source of secondary gas connected to a secondary gas conduit, the secondary gas conduit comprising a secondary gas conduit outlet positioned in close proximity to the liquid conduit outlet.

23. The system of claim 22, wherein the secondary gas is configured to assist in the nebulization process of the liquid.

24. The system of claim 22, further comprising a heater to pre-heat the secondary gas, such that upon mixing of the pre-heated secondary gas with the liquid droplets resulting from the nebulization, the pre-heated secondary gas contributes to the accelerated evaporation of these droplets.

25. The system according to claim 1, further comprising a spray conduit, the spray conduit comprising a spray conduit inlet and a spray conduit outlet, the spray conduit being configured such that the spray conduit inlet encompasses a majority of the volume where nebulization occurs, and such that the stream of nebulizing gas propels the mixture of liquid droplets and aerosol material towards the spray conduit outlet.

26. The system according to claim 1, further comprising an infrared radiation source positioned to heat the liquid droplets produced by the nebulization, thereby contributing to their accelerated evaporation.

27. The system according to claim 1 configured such that at least some aerosol molecular species are produced as a consequence of the nebulization process.

28. The system of claim 27, further comprising an ionization source configured to ionize the aerosol molecular species produced as a consequence of the nebulization process.

29. The system of claim 28, wherein the ionization source is an atmospheric pressure ionization source.

30. The system of claim 29, wherein the ionization method employed by the atmospheric pressure ionization source is selected from the group consisting of: electrospray ionization (ESI), atmospheric pressure chemical ionization (APCI), atmospheric pressure photoionization (APPI), and direct photoionization.

31. The system of claim 28, wherein the ionization source comprises a conductor electrode at a high electric potential, positioned in close proximity to the liquid within the liquid conduit, such that the liquid volume exiting the conduit becomes charged, leading to the aerosol molecular species being charged either by acquiring some of the charges of the liquid during nebulization, or by electrospray ionization if the molecular species are entrapped in the charged liquid droplets resulting from the nebulization process, or by both of these two processes.

32. The system of claim 31, the system being configured such that the aerosol particle concentration at the nebulizing gas conduit outlet is time-dependent, wherein the high electric potential applied to the conductor electrode is pulsed, with such pulsing being synchronized with the temporal arrival of the aerosol particles at the outlet to optimize the charging process.

33. The system of claim 31, further comprising a micro-structured conductor that is in electrical contact with the conductor electrode and positioned such that the liquid is in close contact with the micro-structured features of the micro-structured conductor immediately before nebulization, whereby these micro-structured features increase exposure of the liquid to strong electric fields and consequently facilitate more efficient charging of the liquid exposed to nebulization.

34. The system of claim 28, wherein the ionization source is an atmospheric pressure chemical ionization (APCI) system comprising an auxiliary corona discharge source, through which some of the generated aerosol molecular species transit, thereby acquiring charge via interaction with the corona discharge region.

35. The system of claim 28, wherein the ionization source is an atmospheric pressure photoionization (APPI) system comprising a donor substance that mixes with the aerosol molecular species either during, after, or both during and after the nebulization process, and wherein the APPI system further comprises a UV light source that ionizes the molecules of the donor substance, which in turn transfer their charges to the aerosol molecular species, thereby achieving the ionization of these species.

36. The system of claim 35, wherein the donor substance comprises a gas generated from a donor liquid component introduced into the liquid conduit, which evaporates either during the nebulization process, after it, or at both stages, thereby facilitating the ionization process.

37. The system of claim 35, wherein the APPI system is configured such that the donor gas is introduced as a component of the nebulizing gas mixture through the nebulizing gas conduit outlet, thereby becoming an integral part of the nebulization process.

38. The system of claim 28, wherein the ionization source is an optical source configured to emit radiation that is capable of directly ionizing the aerosol molecular species.

39. The system according to claim 28, further comprising an ion funnel positioned to intercept the generated aerosol ions, such that the electro-magnetic fields of the ion funnel effectively guide a substantial portion of the aerosol ions towards an exit aperture of the ion funnel, thereby enhancing ion collection efficiency.

40. The system of claim 39 further comprising an ion transport conduit, the ion transport conduit comprising an ion transport conduit inlet and an ion transport conduit outlet, where the ion transport conduit inlet is positioned to capture ions emerging from the exit aperture of the ion funnel, facilitating the transfer of the ions towards the ion transport conduit outlet, which is directly connected to the inlet of a mass spectrometer, thereby enabling the efficient injection and subsequent analysis of the aerosol ions.

41. The system according to claim 1 further comprising a gas funnel positioned such that the aerosol material, generated via the nebulization process, is efficiently guided into the gas funnel by the momentum of the nebulizing gas jet, ensuring that a substantial quantity of these particles is effectively directed towards an exit aperture of the gas funnel for subsequent processing or analysis.

42. The system of claim 41 further comprising a transport conduit having a transport conduit inlet and a transport conduit outlet, where the transport conduit inlet is positioned to receive aerosol particles emerging from the exit aperture of the gas funnel, the transport conduit being configured to efficiently transfer these aerosol particles towards the transport conduit outlet, the transport conduit being positioning to facilitate subsequent analysis of the aerosol particles discharged from the gas funnel.

43. The system of claim 42 further comprising an optical source, wherein the transport conduit comprises an optically transparent section, and wherein the optical source is positioned so that its emitted optical radiation passes through the optically transparent section, facilitating optical interaction with the aerosol particle fragments and aerosol molecular species that result from aerosol disintegration as they move through the transport conduit.

44. The system of claim 43 further comprising an optical detector positioned within or adjacent to the transport conduit, the optical detector being configured to detect at least a portion of the optical radiation following its interaction with aerosol particle fragments and aerosol molecular species.

45. The system of claim 43, wherein the system is further configured such that the interaction between the optical radiation from the optical source and the aerosol particle fragments and aerosol molecular species encompasses one or more of the following processes: direct photoionization, optical excitation, or optical scattering.

46. The system according to claim 1, wherein the system is located such that nebulization occurs within an ambient gas environment, wherein said ambient gas is at or near atmospheric pressure.

47. The system of claim 46, the system being configured such that the ambient gas is selected from the group consisting of air, nitrogen, argon, helium, xenon, carbon dioxide, and combinations thereof.

48. The system according to claim 1, wherein the pneumatic nebulizer is configured such that the nebulizing gas is selected from the group consisting of air, nitrogen, argon, helium, xenon, carbon dioxide, and combinations thereof.

49. The system according to claim 1, wherein the pneumatic nebulizer is configured such that a pressure of the nebulizing gas within the nebulizing gas conduit ranges between 1 and 50 bars.

50. The system according to claim 1, wherein the pneumatic nebulizer is configured such that the volume flow rate of the nebulizing gas within the nebulizing gas conduit is within the range of 0.1 to 30 liters per minute.

51. The system according to claim 1 wherein the liquid source unit is configured such that a liquid flow rate within the liquid conduit is maintained within the range of 1 to 3000 μL/min.

52. The system of claim 2, wherein the liquid solvent is selected from the group consisting of water, organic solvent, alcohol, methanol, ethanol, isopropanol, acetone, acetonitrile, dimethyl sulfoxide, chloroform, benzene, hexane, ethyl acetate, toluene, ionic liquid, and their mixtures.

53. The system according to claim 1, wherein the liquid source unit is configured such that the liquid emanating from the liquid source contains a chemical agent that advantageously reacts with at least a portion aerosol particle fragments and aerosol molecular species either during the nebulization process, within the liquid droplets resulting from the nebulization, or in both states.

54. The system of claim 53, wherein the chemical agent comprises a molecular label that preferentially attaches to a specific subset of the aerosol molecular species, thereby facilitating the analysis of that subset.

55. The system according to claim 54 wherein the molecular label is selected from the group consisting of: enzymes, biotin, Halo tags, SNAP tags, nanoparticles, optical fluorescent labels, Raman labels, luminescent labels, radioactive isotopes, gold particles, magnetic labels, quantum dots, metal ions, or a combination thereof.

56. A system comprising a first sub-system and a second sub-system, each sub-system being configured according to claim 1, wherein the first sub-system is positioned relative to the second sub-system such that the aerosol material outputted from the first sub-system is utilized as the input aerosol material for the second sub-system.

57. The system of claim 1, further comprising a laser system capable of generating sample aerosol particles through laser ablation of a sample, wherein the aerosol particles are ejected from the sample as a plume.

58. The system of claim 57, wherein the laser system operates at a wavelength or plurality of wavelengths ranging from 100 nm to 12 μm.

59. The system of claim 57, wherein the laser output of the laser system is pulsed, with the duration of each laser pulse ranging between 10 femtoseconds and 1 millisecond.

60. The system of claim 57, wherein the laser system is a component of a surgical device.

61. The system of claim 57, wherein the laser system is a component of a tissue imaging device.

62. The system of claim 57, further comprising an imaging device configured such that prior to laser ablation, the sample is imaged using the imaging device to provide data that is employed for the selection of the laser sampling spot or to furnish additional information about the sample, augmenting the analysis of the sample aerosol particles.

63. The system of claim 62, wherein the imaging device is a vibrational imaging device.

64. The system of claim 63, wherein the vibrational imaging device employs an imaging modality selected from the group consisting of Raman imaging, stimulated Raman scattering (SRS) imaging, coherent anti-Stokes Raman scattering (CARS) imaging, infrared imaging, and combinations thereof.

65. The system of claim 57, further comprising a photoacoustic imaging device configured such that prior to laser ablation, the sample is imaged using the photoacoustic imaging device to provide data that guides the determination of the laser sampling spot location or to furnish additional information about the sample, supplementing the analysis of the sample aerosol particles.

66. The system of claim 57, further comprising an optical coherence tomography (OCT) device configured such that prior to laser ablation, the sample is imaged using the optical coherence tomography (OCT) device to provide data that assists in determining the location of the laser sampling spot or to offer additional information about the sample, complementing the analysis of the sample aerosol particles.

67. The system according to claim 1 further comprising an electrosurgical device configured to generate the sample aerosol particles via contact with a biological tissue.

68. The system of claim 1, further comprising a mass spectrometer positioned to receive ions generated from the aerosol particles, enabling the mass spectrometer to analyze the ions and thereby provide information about the chemical composition of the sample aerosol particles collected by the aspirator device.

69. The system of claim 68, wherein the mass spectrometer includes an ion drift tube filled with a buffer gas, the ion drift tube comprising an ion drift tube inlet and an ion drift tube outlet, an ion trap in ion communication with the ion drift tube inlet, and an ion gating mechanism located at the ion drift tube outlet to regulate ion exit, wherein the ion trap accumulates aerosol ions and periodically releases them into the ion drift tube, and wherein the ion transition time through the ion drift tube is dependent on ion gas mobility within the buffer gas; and wherein the mass spectrometer measures the mass-to-charge ratio of ions exiting the ion drift tube.

70. A method for analyzing aerosol particles, the method comprising:
employing the system of claim 57 to obtain aerosol particles that have been at least partially disintegrated; and
employing an analytic system to detect and analyze the at least partially disintegrated aerosol particles.

71. The method according to claim 70 wherein the analytic system comprises a mass spectrometer.

72. The method according to claim 70, wherein the aerosol particles are collected by the aspirator device, and wherein the at least partially disintegrated aerosol particles comprise ions, and wherein these ions are subsequently analyzed by a mass spectrometer.

73. The method according to claim 70, wherein the diameters of at least some of the sample aerosol particles are within the range of 1 nanometer to 100 micrometers.

74. The method according to claim 70 wherein the sample aerosol particles are generated from in vivo biological tissue.

75. The method according to claim 70 wherein the sample aerosol particles are generated from the ex vivo biological tissue.
